(19) United States
(12) Patent Application Publication
(10) Pub. No.: US 2019/0143119 A1
(43) Pub. Date: May 16, 2019

(54) Systems and Methods for Brain Stimulation

(71) Applicant: Duke University, Durham, NC (US)

(72) Inventors: Kafui Dzirasa, Durham, NC (US); Neil M. Gallagher, Durham, NC (US)

(21) Appl. No.: 16/087,792

(22) PCT Filed: Mar. 28, 2017
(86) PCT No.: PCT/US2017/024487

§ 371 Date: Sep. 24, 2018

Related U.S. Application Data
(60) Provisional application No. 62/314,371, filed on Mar. 28, 2016.
(60) Provisional application No. 62/337,464, filed on May 17, 2016.

(51) Int. Cl. A61N 1/36; A61N 1/05

(52) U.S. Cl. CPC A61N 1/36096; A61N 1/0529; A61B 5/72; A61N 1/36146; A61N 1/36135

(57) ABSTRACT

A variety of pathological behavioural and/or psychological states can be caused and/or characterized by changes in the degree of functional coupling between regions of the brain relative to a baseline degree of coupling. These pathological behavioural and/or psychological states may include bipolar disorder, mood disorders, PTSD, anxiety disorders, schizophrenia, or autism. Such changes may be caused by experiences and/or genetic factors. To treat such pathological behaviour and/or psychological states, the degree of functional coupling between particular regions of the brain may be increased. An increase in the functional coupling between first and second brain regions may be caused by providing repeated excitatory stimuli to the first brain region in phase with oscillatory patterns of activity in the second brain region.

### CROSS-REFERENCE TO RELATED APPLICATIONS

This application claims the benefit of and incorporates by reference the content of U.S. Provisional App. No. 62/314,371, filed Mar. 28, 2016, and U.S. Provisional App. No. 62/337,464, filed May 17, 2016.

### GOVERNMENT LICENSE RIGHTS

This invention was made with government support under Federal Grant No. R01MH099192 awarded by the National Institute of Mental Health. The government has certain rights in the invention.

### BACKGROUND

Challenging experiences promote cellular adaptations across many cortical and limbic regions and/or other regions of the brain including prelimbic cortex, prefrontal cortex, nucleus accumbens, amygdala, medial dorsal thalamus, hippocampus, and the ventral tegmental area. Multiple studies have linked such cellular changes to the emergence of behavioral dysfunction. For example, stress-induced changes in hippocampal and striatal morphology and excitatory transmission have been implicated in mediating mala.daptive behavioral changes including deficits in learning, decision making, and hedonic behavior. Similarly, multiple changes in gene expression, cellular firing, and circuit function in prefrontal cortex have been linked to deficits in social behavior and anxiety related behavior. While, in principle, these and many other heterogeneous cellular/molecular adaptations converge to disrupt the function of mesoscale networks that regulate normal emotional behavioral processes, studies that directly probe the link between adaptations at the neural network level and the emergence of behavioral pathology have been limited due to the sparsity of tools to directly quantify neural networks activity with high spatiotemporal resolution.

Such pathological states may be addressed by attempting to compensate and/or reverse relevant changes in the function of the brain. Such effects may, in some cases, be achieved through the application of pharmaceuticals to increase or decrease the activity of a brain regions, to modulate the effect of a first brain region on one or more further brain regions (e.g., by altering the behavior of neurotransmitters, receptions, and/or cells projecting between the brain regions). Additionally or alternatively, electrodes or other devices may be used to directly stimulate activity and/or to reduce activity in one or more brain regions.

### SUMMARY

In certain pathological states, the strength of connections between different regions of the brain may be functionally diminished. Such a decrease in functional connectivity of brain regions may be due to an experience (e.g., due to an individual trauma, due to repeated traumas and/or chronic exposure to stressors), due to the activity of one or more genes, or due to some other process or mechanism. Such pathological states and/or signs or symptoms associated with such pathological states may be decreased or abolished by inducing an increase in the functional connectivity between such different brain regions. Such an increase in functional connectivity may be induced by providing repeated excitatory stimuli to one of the brain regions in-phase with ongoing oscillatory activity in a further one of the brain regions. This could include detecting a phase of oscillatory activity in the further one of the different brain regions and providing, at a specified offset from the detected phase of the oscillatory activity, excitatory stimulus to the first one of the different brain regions.

In an aspect, a system is provided. The system includes a stimulator, a waveform generator communicatively coupled to the stimulator, a data acquisition system, and a controller having a memory and at least one processor. The at least one processor is configured to execute instructions stored in the memory so as to carry out operations. The operations include receiving, via the data acquisition system, information indicative of an oscillatory signal from a first brain region. The operations also include determining, based on the received information, a reference phase of the oscillatory signal. The operations also include generating, by the waveform generator, at least one excitatory stimulus. An onset of the generated at least one excitatory stimulus is based on the reference phase of the oscillatory signal. The operations yet further include, in response to the generated at least one excitatory stimulus, causing the stimulator to provide stimulation to a second brain region.

In an aspect, a method is provided. The method includes detecting, from a first brain region, an oscillatory signal. The method also includes determining, based on the detected oscillatory signal, a reference phase of the oscillatory signal. The method additionally includes providing, to a second brain region, at least one excitatory stimulus, wherein an onset of the at least one excitatory stimulus is based on the reference phase of the oscillatory signal.

In an aspect, a system is provided. The system includes various means for carrying out the operations of the other respective aspects described herein.

These as well as other embodiments, aspects, advantages, and alternatives will become apparent to those of ordinary skill in the art by reading the following detailed description, with reference where appropriate to the accompanying drawings. Further, it should be understood that this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF THE FIGURES

FIG. 1 illustrates a schematic diagram of a system, according to an example embodiment.

FIG. 2 illustrates elements of a brain sensing and stimulation device, according to an example embodiment.

FIG. 3 illustrates a human brain, according to an example embodiment.

FIG. 4A illustrates an LFP signal, according to an example embodiment.

FIG. 4B illustrates an LFP signal and an excitatory signal, according to an example embodiment.

FIG. 4C illustrates an excitatory signal, according to an example embodiment.

FIG. 5 illustrates a method, according to an example embodiment.

FIG. 6A illustrates signals recorded during a tail suspension test.

FIG. 6B illustrates neural firing signals and peri-event histograms recorded from a variety of different brain regions during a tail suspension test.

FIG. 6C illustrates firing rates recorded during a tail suspension test.

FIG. 6D illustrates relationships between firing rates and behavioral activity in a variety of different brain regions.

FIG. 7 illustrates peri-event histograms recorded from a variety of different brain regions during a tail suspension test.

FIG. 8A illustrates histograms of neural firing during a tail suspension test.

FIG. 8B illustrates histograms of neural firing during an open field test.

FIG. 8C illustrates immobility time and distance traveled during a tail suspension test and an open field test.

FIG. 8D illustrates movement function of a variety of different brain regions during a tail suspension test.

FIG. 8E illustrates changes in immobility time across different mouse genetic variants.

FIG. 8F illustrates neural activity in two different brain regions across days of application of a tail suspension test and across different mouse genetic variants.

FIG. 9A illustrates relationships between behavioral activity and recorded neural signals during a tail suspension test.

FIG. 9B illustrates changes in response during a tail suspension test across multiple days.

FIG. 10A illustrates signals recorded from two brain regions and properties of oscillatory signals of the recorded signals.

FIG. 10B illustrates phase offsets, power, and coherence of signals recorded from two brain regions.

FIG. 11A illustrates the location of two different brain regions and a schematic of connections between the two brains regions when the brain regions are being interacted with by a stimulating system.

FIG. 11B illustrates elements of a brain stimulating system and signals that could be detected by and/or generated by such a system.

FIG. 11C illustrates effects of brain stimulation on behavior during a tail suspension test.

FIG. 11D illustrates effects of brain stimulation on behavior during a tail suspension test.

FIG. 11E illustrates effects of brain stimulation on behavior during a tail suspension test.

FIG. 11F illustrates effects of brain stimulation on behavior during a tail suspension test.

FIG. 11G illustrates effects of three varieties of brain stimulation on behavior during a tail suspension test.

FIG. 11H illustrates proposed relationships between the activities of different brain regions.

FIG. 12A illustrates power and coherence of signals recorded from different brain regions.

FIG. 12B illustrates relationships between the activities of different brain regions in a wild type genetic mouse variant.

FIG. 12C illustrates relationships between the activities of different brain regions in a ClockΔ19 genetic mouse variant.

FIG. 12D illustrates proposed relationships between the activities of different brain regions in a wild type genetic mouse variant.

FIG. 12E illustrates proposed relationships between the activities of different brain regions in a ClockΔ19 genetic mouse variant.

FIG. 13A illustrates regions of a brain.

FIG. 13B illustrates the timing of an experimental protocol.

FIG. 13C illustrates the coherence between different brain regions before and after a treatment.

FIG. 14 illustrates mean neural firing rates recorded from different brain regions during different sessions of a tail suspension test.

FIG. 15 illustrates a relationship between immobility times determined using an automated method and immobility times determined manually.

FIG. 16 illustrates a relationship between a change in immobility time between sessions of a tail suspension test and the duration of the immobility time during an initial session of the tail suspension test.

FIG. 17 illustrates the location of electrodes in a variety of different brain regions.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features. Other embodiments can be utilized, and other changes can be made, without departing from the scope of the subject matter presented herein.

Thus, the example embodiments described herein are not meant to be limiting. Aspects of the present disclosure, as generally described herein, and illustrated in the figures, can be arranged, substituted, combined, separated, and designed in a wide variety of different configurations, all of which are contemplated herein.

Further, unless context suggests otherwise, the features it ustrated in each of the figures may be used in combination with one another. Thus, the figures should be generally viewed as component aspects of one or more overall embodiments, with the understanding that not all illustrated features are necessary for each embodiment.

### I. Overview

A variety of psychological disorders or conditions may be caused by a variety of changes in the structure and/or functioning of elements of the human brain. These changes may be induced by an intrinsic process (e.g., by a genetic predisposition to a psychological disorder), by a trauma (e.g., head trauma, surgery, stroke, hypoxia), by one or more experiences a traumatic experience, chronic exposure to stressful conditions or environments), or by some other mechanism(s). These changes can include cell death, changes in the intrinsic excitability of one or more cells or brain regions, changes in the functional connectivity between cells and/or brain regions (e.g., due to loss of axons, a change in the amount of neurotransmitter emitted from an axon, a change in the functional effect of a cell receiving an amount of neurotransmitter from an axon), changes in the neurophysiological environment (e.g., changes in glial function, changes in the amounts of hormones, neurotransmitters, or other substances in the brain, a change in pressure or some other changes in the structure and/or functioning of the brain.

Treatment of such psychological disorders or conditions may include a variety of interventions to reverse such changes and/or compensate for the effects of such changes. Treatments may include talk therapy, pharmaceutical intervention, surgical intervention, electrical stimulation, or some other procedures or therapies. Treatments may act to compensate for changes underlying a psychological disorder or condition (e.g., by increasing the availability of a neurotransmitter by reducing the rate of reuptake of the neurotransmitter, by increasing the intrinsic activity of a brain region, by blocking the activity of a brain region and/or blocking efferents from the brain region) and/or to reverse the changes underlying the psychological disorder or condition.

It can be possible to treat some psychological disorders by reversing a change in functional connectivity between two (or more) regions of the brain or to otherwise increase the degree of interaction between such two (or more) brain regions. For example, certain psychological disorders or conditions, including post-traumatic stress disorder (PTSD), autism, schizophrenia, and bipolar disorder could be treated by changing a degree of interaction between brain regions. This treatment could include providing, in sync with periodic activity in a first brain region, repeated excitatory stimulation to a second brain region. This stimulation of a ‘downstream’ brain region, synchronized to the activity of an ‘upstream’ brain region (that is, an ‘upstream’ region that projects axons or otherwise provides efterent stimulus to the ‘downstream’ brain region stimulated by the treatment), may act to induce or otherwise strengthen a functional coupling or connection between the upstream and downstream brain regions or act by some additional or alternative mechanism(s) to treat the causes and/or symptoms of a psychological disorder or condition.

Stimulating a second brain region, based on the detected oscillatory or otherwise repeating activity of a first brain region, could involve providing electrical signals (e.g., pulses or other waveforms of current and/or voltage), optical stimulation (e.g., to neurons that are sensitive to optical stimulation and/or that have been made sensitive by some intervention), chemical stimulation (e.g., by emission of timed amounts of a stimulating neurotransmitter or other substance), or providing some other excitatory stimulus in a manner that is timed according to the timing or other information related to detected activity of the first brain region. Stimuli could be provided at a particular time or phase reference position relative to ongoing detected oscillatory activity of the first brain region, across a range of phases relative to the detected activity of the first brain region, or according to some other method or pattern. Provided stimuli could include square pulses, triangular pulses, or some other waveforms or patterns of stimulation.

A variety of different physical variables could be detected from the first brain region to determine a timing of the stimuli provided to the second brain region. An electrical signal (e.g., electrical activity of a single cell, multiple cells, or the averaged activity of an entire region of the brain), an optical signal (e.g., of neurons that intrinsically emit light or change some optical property related to activity or that have been caused to have such an activity-dependent optical property by genetic modification or other means), a chemical signal (e.g., neurotransmitter levels, levels of oxygen or other metabolites), a temperature signal, or some other signals related to the ongoing activity of the first brain region could be detected. A phase or other information (e.g., a frequency, a coherence between two or more bands of frequencies) about an oscillatory pattern in the detected signal (e.g., a local field potential (LFP)) could be determined and used to control the timing of provided excitatory stimuli.

Systems and devices configured to effect the therapies and methods described herein (e.g., to detect timing information about oscillatory or otherwise repeating activity of a first brain region and to provide excitatory stimuli to a second brain region based on the detected timing information) could include implanted and/or non-implanted elements. Implanted elements could include electrodes, optical fibers, leads extending beneath the skin between elements of the device or system, leads or connectors traversing the skin to permit connections between implanted and non-implanted elements, controllers, batteries, communications or power transfer coils, or other elements. Non-implanted elements could include wearable electrodes or devices (e.g., an array of electroencephalogram (ERG) electrodes configured to be worn as a cap), coils for the delivery of transcranial magnetic stimulation (TMS), magnetic resonance imagers (MRI), magnetoencephalography (MEG) equipment, control interfaces (e.g., for use in setting parameters of operation of the system by a clinician or other user), power sources, or other elements. Implanted and non-implanted elements may be in wireless or wired communication (e.g., an implanted device that is configured to detect activity in a first brain region could communicate phase or other timing information about the detected activity to a TMS stimulator that is located outside of the body) and/or may be in communication with other systems (e.g., a computer in a physician's office).

The devices, systems, and methods described herein are not limited to the detection of activity in a single brain region and providing excitatory stimuli, based on the detected activity, to a single further brain region. Stimuli could be provided to one or more brain regions based on the timing or other properties of activity detected in one or more brain regions. Stimuli could be provided and/or activity detected in a brain region on one side of the brain (unilaterally) or on both sides of the brain (bilaterally). Other patterns of detection from one or more regions and providing stimuli to one or more further regions and/or to the regions from which the activity is detected are anticipated by the inventors.

### II. Example Systems

A variety of devices and systems could be configured to detect oscillatory or otherwise repeating signals from one or more brain regions and to provide, to one or more further brain regions, excitatory stimuli based on a determined phase or other timing information that is present in the detected signals according to a treatment for a psychological disorder or condition or according to sonic other application.

Such systems could include elements that are implanted within a body (e.g., within a brain) and/or elements that are disposed outside of the body (e.g., electrodes or other elements that may be removably mounted to the skin of the body). Elements that are implanted within a body could be operably coupled to elements outside of the body (e.g., mounted to a surface of the body) by cabling, connectors, or other means passing through the skin. Additionally or alternatively, elements of such systems could be wirelessly coupled, e.g., by coils, antenna, or other means for transmitting and/or receiving wireless communications and/or wireless power. Such systems could be configured for chronic use (e.g., to provide stimulation over a protracted period of time) or for acute and/or periodic use.

Such systems could be configured to detect a variety of different signals related to oscillatory or otherwise repeating patterns of activity in one or more brain regions. For example, such systems could be configured to detect electrical current or voltages, electrical fields, magnetic fields, chemical concentrations or gradients, acoustical waves, temperatures or temperature gradients, the intensity or wavelength of light emitted from a brain region (e.g., in response to illumination or some other applied energy or generated internally by the brain region), or some other physical variable related to the activity of a brain region. Furthermore, such systems could be configured to provide a variety of different types of stimuli to excite a brain region. For example, such systems could be configured to apply electrical currents or voltages, time-varying magnetic fields, radio frequency waves or fields, light (e.g., infrared light, ultraviolet light, or visible light to stimulate an optically-sensitive substance present in a brain region), chemical substances (e.g., neurotransmitters), acoustical waves, or other energies or substances that can excite activity in elements (e.g., cells, axons) of the brain.

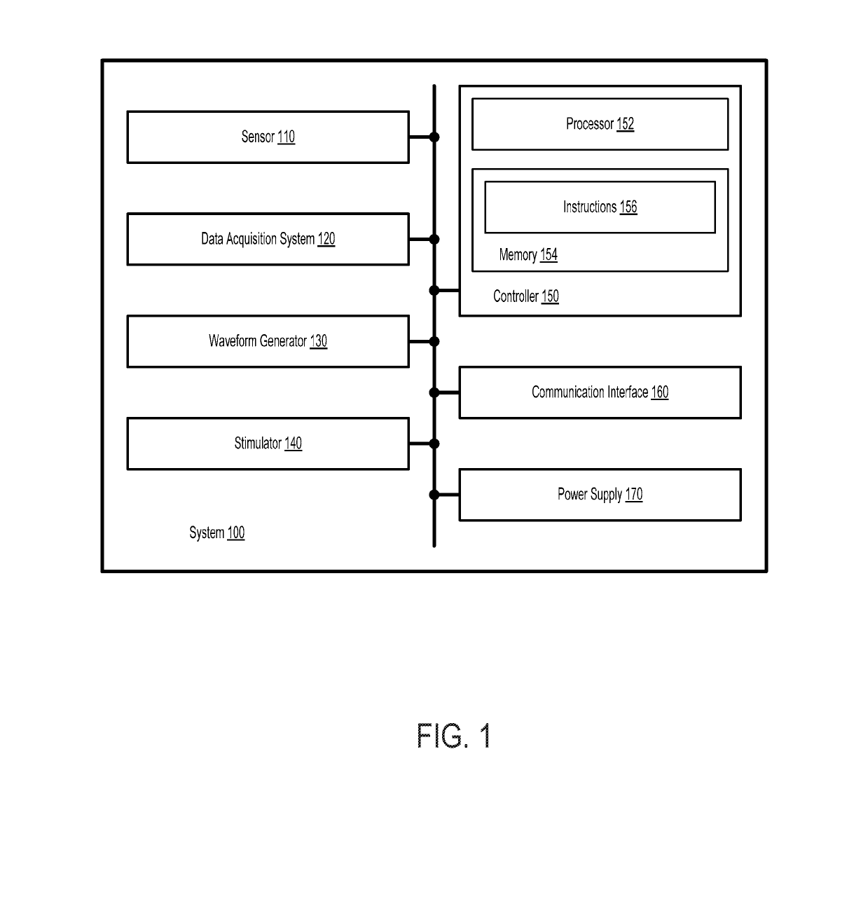

FIG. 1 illustrates a schematic diagram of a system 100, according to an example embodiment. System 100 includes at least one sensor 110, a data acquisition system 120, a waveform generator 130, and at least one stimulator 140. System 100 also includes a controller 150, a communication interface 160, and a power supply 170. The sensor 110 is configured to detect an oscillatory signal related to the activity of a first brain region (e.g., to detect a local field potential, an electroencephalogram, an electrical field generated by the electrical activity of one or more neurons, or some other electrical voltage or current related to the oscillatory or otherwise repeating electrical activity of the first brain region). The stimulator 140 is configured to provide an excitatory stimulus to a second brain region (e.g., an electrical voltage and/or current, an optical signal at a wavelength specified to excite an optically sensitive protein, channel, or other element(s) of the second brain region).

It is understood that some or all elements of system 100 may be implantable in a patient. Furthermore, at least some elements of system 100 may remain external to, and/or may be worn by, the patient. Further, individual illustrated components (e.g., the sensor 110, the stimulator 140, the controller 150) may be distributed and/or duplicated across multiple elements of the system 100. For example, the sensor 110, the data acquisition system 120, and elements of the controller 150 may be disposed in an implanted device while the waveform generator 130, the stimulator 140, and other elements of the controller 150 may be disposed in an external device that is configured to be mounted to or otherwise maintained in position relative to the brain of a person, outside of the body of the person. In such an example, the implanted device could be configured to detect a phase of an LFP in a first brain region and to transmit information about the detected phase (e.g., by transmitting the detected LFP signal and/or by determining a reference phase of LFP and transmitting timing information or other information about the reference phase) to the external device. The external device could then operate to provide excitatory stimulus to a second brain region, in the form of transcranial magnetic stimulation pulses, to a second brain region based on the received reference phase information (e.g., such that the excitatory stimulus is provided at a set time or range of times relative to the reference phase).

The sensor 110 is configured to transduce or otherwise facilitate the detection of oscillatory or otherwise repeating signals in a first region of the brain. These signals could be detected from a small region of the brain (e.g., action potentials and/or extracellular signals from a single neuron, local field potentials or other oscillatory signals generated by a population of hundreds or thousands of neurons in a volume of the brain) or from a larger region of the brain (e.g., an electroencephalogram from the activity of neurons across centimeters or more of the surface of the brain).

The sensor 110 could be configured to detect a physical variable related to the oscillatory signal of interest. The physical variable could be an electrical voltage, current, or field (e.g., the sensor 110 could include one or more electrodes), a magnetic field (e.g., the sensor 110 could include a superconducting quantum interference device (SQUID) or some other sensitive magnetometer), a light emitted from an intrinsically light-emitting element of the brain (e.g., the sensor 110 could include a photodetector configured to detect light emitted from genetically modified neurons), a light emitted from an element of the brain in response to illumination or emission of some other energy from the sensor 110 to the brain (e.g., the sensor 110 could include a photodetector configured to detect a light emitted from a calcium-sensitive dye or fluorophore in response to illumination by the sensor 110), a chemical concentration (e.g., the sensor 110 could include an amperometric or otherwise configured sensor configured to detect a concentration of a neurotransmitter, a protein, a metabolite, or some other chemical species), or some other physical variable related to an oscillatory signal of interest or related to some other signal related to the activity of a region of interest of the brain.

The sensor 110 could be configured to detect such signals from outside of the brain. This could include detecting an electrical and/or magnetic field outside of the skull (e.g., using a magnetoencephalogram), detecting an electrical field and/or current on or within the scalp (e.g., using one or more electrodes disposed on or within the scalp to detect an electroencephalogram), detecting an electrical field and/or current at one or more locations within or beneath the skull (e.g., using one or more electrodes disposed on the dura and/or beneath the dura on the surface of the brain or within the scalp to detect an electroencephalogram and/or a local field potential), or detecting some other signal of interest outside of the brain. Additionally or alternatively, the sensor 110 could be configured to detect signals within the brain. This could include detecting an electrical field and/or current within the cortex of the brain (e.g., using one or more penetrating electrodes), detecting light emitted and/or reflected from within the cortex of the brain (e.g., using a penetrating device that includes an optical fiber or other means for detecting such a light), detecting an electrical field and/or current within a deep structure of the brain (e.g., using one or more electrodes disposed on a lead penetrating from the surface of the brain to the deep structure), detecting light emitted and/or reflected from a deep structure of the brain (e.g., using a device that penetrates from the surface of the brain to the deep structure and that includes an optical fiber or other means for detecting such a light), or detecting some other signal(s) from within the brain.

The sensor 110 may include leads, optical fibers, or other means for transmitting a signal of interest from a brain region that is generating the signal to the data acquisition system 120. For example, the sensor 110 could include an optical fiber, lenses, and/or other optical elements configured to transmit light emitted from a brain region (e.g., light emitted intrinsically from the brain region and/or emitted from the brain region in response to illumination or to some other applied energy) to a photodetector and/or other light detecting means of the data acquisition system 120. Additionally or alternatively, elements of the data acquisition system 120 may be disposed proximate to the brain region generating such a signal. For example, amplifiers, filters, analog-to-digital converters, photodetectors, light emitters, integrated circuits, or other electronic elements may be disposed on the backside of one or more surface electrodes, proximate the tip of a device configured to penetrate from the surface of the brain to a deep structure of the brain, or at some other location proximate a brain region of interest and/or elements of the sensor 110 (e.g., electrodes, optical fibers, lenses, chemical transducers) configured to detect an oscillating signal from the brain region.

Sensor 110 may include one or more electrodes configured to conduct electrical current and/or measure a local field potential (LFP) within a brain region. An LFP may be a signal generated by one or more neurons proximate to an electrode of the sensor 110. In some embodiments, the LFP signal may provide information about physiology such as: synaptic currents, neuronal connectivity, average neural activity, a degree of coherence and/or synchronization of activity of a population of neurons, and/or neural interaction within a given brain region. The LFP signal may include oscillating signals having frequencies between 3-6 Hz, but LFP signals that include components at other frequencies are possible.

In an example embodiment, sensor 110 may include an implantable electrical lead that includes wires configured to be temporarily or permanently implanted proximate to a brain region. Sensor 110 may include a bipolar, tripolar, or quadripolar lead that includes a plurality of insulated wires housed within an insulating material, such as polyimide, polyethylene, or polyurethane. Each of the wires may terminate at an exposed electrode located near a tip portion of the lead. Each of the exposed electrode locations may be disposed in a tetrode arrangement or according to some other pattern in space. The exposed electrodes may include titanium, gold, poly(3,4-ethylenedioxythiophene) polystyrene sulfonate, or another conductive, biocompatible material. At least one of the exposed electrodes may include a reference electrode. One or more of the other exposed electrodes may be sensing electrodes. In other embodiments, the reference and sensing electrodes may be interchangeable based on, for example, user preference.

In some embodiments, sensor 110 may include a cylindrical lead approximately 1-2 mm in diameter and 40 cm in length. The lead may include a tungsten wire or another stiff material to ease targeting and delivery of the lead to a proper location within the brain (e.g., a deep structure of the brain like an element of the hippocampus, striatum, thalamus, tegmentum, hypothalamus, limbic system, or some other deep structure(s) of the brain) and/or may be driven by a removable stylus. Sensor 110 may include circuitry configured to detect a voltage between (and/or current passing through) two electrodes of sensor 110. For example, the circuitry may include a low-pass filter having a cutoff frequency between 100 Hz and 1 kHz. Furthermore, the circuitry may include one or more amplifier stages and/or noise-reduction stages configured to amplify a detected signal (e.g., an LFP signal) and/or improve signal to noise ratio of the signal. In some embodiments, the sensor 110 may include one or more optical fibers, lenses, optical filters, gratings, or other optical elements configured to transmit light emitted from a deep region of the brain, via the cylindrical lead, to a photodetector.

In some embodiments, at least a portion of sensor 110 may be implanted via a burr hole in a patient's skull. Upon proper delivery/placement of sensor 110, a portion of sensor 110 may be anchored to the patient's skull. Sensor 110 may be communicatively coupled to data acquisition system 120 via a wireless or wired connection, which may include communication interface 160.

Data acquisition system 120 is configured to detect, using the sensor 110, an oscillatory signal of interest (e.g., an LFP, an EEG, a firing rate of one or more neurons) that may be related to the activity of a first brain region. The data acquisition system 120 may include a single or multi-channel recording system; that is, the data acquisition system 120 may be configured to detect oscillatory signals (or other signals relating to such oscillatory signals) from one or more sensors and/or elements of a sensor (e.g., one or more electrodes of an electrode array, one or more electrodes of a tetrode). In an example embodiment, data acquisition system 120 may receive electrical signals from sensor 110. The electrical signals may include information indicative of an LFP in a first brain region. In such a scenario, data acquisition system 120 may convert an analog voltage waveform to a digital signal. For example, data acquisition system 120 may include one or more voltmeters or analog-to-digital converter circuits, which may provide information indicative of one or more LFP or excitatory signals in units of volts. In some examples, the data acquisition system may be configured to operate one or more light emitters, piezoelectric transducers, or other elements configured to emit an energy into the first brain region (e.g., light energy, acoustical energy) to cause the first brain region to emit light having an oscillatory intensity or other property related to activity in the first brain region (e.g., to cause a calcium-sensitive dye or fluorophore to emit light having an intensity related to an oscillatory calcium concentration within one or more neurons in the first brain region) or to cause the first brain region to generate some other oscillatory signal of interest.

Data acquisition system 120 may include one or more computers, such as a digital signal processing system. Additionally or alternatively, data acquisition system 120 may be connected to one or more computers via communication interface 160. In some embodiments, data acquisition system 120 may provide one or more clock and/or trigger signals to another element of system 100, such as waveform generator 130. Data acquisition system 120 may be configured to record and store signals locally and/or in the memory 154 of controller 150.

Waveform generator 130 could be configured to generate a variety of different signals and/or waveforms to facilitate the generation of one or more excitatory stimuli by the stimulator 140. In some examples, the waveform generator 130 may include an arbitrary waveform generator (AWG), which may be configured to provide various electrical waveforms. Additionally or alternatively, the waveform generator 130 may be configured to generate a stimulus waveform that is limited with respect to one or more parameters, e.g., to produce a stimulus waveform that includes a set number of pulses, each having a set pulse shape (e.g., a square waveform), pulse width, and amplitude, while being able to control the frequency of the pulses and to control the timing of the stimulus waveform relative to a detected oscillatory signal (e.g., relative to a reference phase or other timing information determined based on a signal detected using the sensor 110). In such examples, the waveform generator 130 may accept, as an input, a clock and/or trigger signal and could be configured to provide a set stimulus waveform (e.g., a gamma pulse of comprising three separate square pulses having respective pulse widths, amplitudes, inter-pulse intervals, or other properties) at a set latency in response to receiving such a clock or other trigger signal. The waveform generator 130 may provide electrical voltage or current waveforms, patterns of emitted light intensity, sequences of digital codes (e.g., representing current amplitudes to be applied to the second brain region using the stimulator 140), or some other outputs that could cause the stimulator 140 to provide at least one excitatory stimulus to the second brain region.

In an example embodiment, waveform generator 130 may receive a trigger pulse from data acquisition system 120 and/or controller 150. In response to the trigger pulse, waveform generator 130 may provide an analog voltage waveform as an output to stimulator 140. For example, the analog voltage waveform may include a gamma pulse. A gamma pulse is an excitatory waveform that includes at least two pulses having a frequency and/or inter-pulse interval specified to correspond to a gamma frequency of a brain region being stimulated and/or specified such that activity induced in the stimulated brain region, in response to reception of the gamma pulse, has frequency content across a broad range of frequencies. Each gamma pulse may include three square wave pulses. In the case where stimulator 140 includes a light source, the pulse amplitude of the square wave may be five volts or another typical voltage for enabling the light source. In the case where stimulator 140 is configured to provide an electrical excitatory stimulation, the square wave amplitude may be different, e.g. 10-100 mV.

While examples herein include square wave pulses, the voltage, current, light, or other waveform provided to stimulator 140 by the waveform generator 130 need not be a square wave. In other words, the voltage waveform provided to stimulator 140 may include a sine wave, a sawtooth/ramp, a raised Gaussian, one or more paired, balanced pulses (e.g., to balance an amount of charge injected into tissue of the brain using an electrode), or another type of waveform shape. Furthermore, while sonic of the example gamma pulses herein include three individual pulses with similar amplitudes, pulse widths, and inter-pulse intervals, gamma pulses having different properties are possible. For example, voltage waveforms that include more or fewer pulses are contemplated. Furthermore, voltage waveforms that include pulses with differing amplitudes, pulse widths, and/or inter-pulse intervals are contemplated.

The stimulator 140 may be configured to provide electrical, optical, chemical, acoustical, or some other variety of excitatory stimuli to a second brain region according to one or more outputs of the waveform generator 130. The stimulator 140 could include light emitters, electrodes, piezoelectric transducers, chemical transducers, or other tissue-stimulating elements. The stimulator 140 could include amplifiers, filters, transducers, digital-to-analog converters, buffers, or other elements configured to convert, condition, or otherwise modify signals generated by the waveform generator 130 such that the stimulator 140 provides at least one pulse of excitatory stimulus to a second brain region. The stimulator 140 could include blocking capacitors, resistors, fuses, clamping diodes, or other elements to prevent the application of deleterious stimuli to the second brain region (e.g., to limit a maximum current and/or voltage applied, by electrodes of the stimulator 140, to the brain). The stimulator 140 could include wires, optical fibers, lenses, leads, waveguides, or other elements configured to transmit a generated electrical voltage or current, a light, an acoustical energy, or some other stimulus signal to the second brain region from an emitting element of the system 100 (e.g., from a light emitter, electrical buffer or amplifier, or other element(s) of the stimulator 140 and/or waveform generator 130).

In an example embodiment, the brain region receiving the electrical stimuli may be different from the first brain region from which the LFP or other oscillatory signal is obtained. In such a scenario, stimulator 140 may include one or more optical fibers, wires connected to one or more electrodes, or other elements that may be proximate to the second brain region. The stimulator 140 may provide voltage waveforms, light intensity waveforms, of other signals generated by waveform generator 130 to the second brain region. In some embodiments, stimulator 140 may amplify, denoise, or otherwise adjust voltage waveforms provided by waveform generator 130 before providing such waveforms to the second brain regions. Accordingly, stimulator 140 may include circuitry such as filters, amplifiers, etc.

Stimulator 140 may be similar or identical to sensor 110. For example, stimulator 140 and sensor 110 could bath include respective one or more electrodes configured to provide an electrical interface with tissues of the brain. Furthermore, stimulator 140 may be incorporated into at least some elements of sensor 110. In an example embodiment, stimulator 140 and sensor 110 may be combined into a single implantable device. Furthermore, by time division, frequency division, or another controllable method, one or more of the electrodes of the implantable device may act as either a sensing electrode or a stimulating electrode. For example, an electrode of the stimulator 140 could be used both to provide an excitatory electrical stimulus to a second brain region and to detect an electrical signal from the second brain region. Such an electrical signal could include an LFP or some other signal that could be used to control some aspect of operation of the system 100, to assess the efficacy and/or progress of a treatment provided using the system 100, or to facilitate some other application. For example, a detected LFP could be used to determine a frequency of gamma activity in the second brain region and to control an inter-pulse interval of gamma pulses provided to the second brain region using the stimulator 140. In other embodiments, stimulator 140 may take the form of an implantable device that is separate with respect to sensor 110.

In an alternative embodiment, stimulator 140 may be configured to provide optical stimuli to the second brain region. For example, the second brain region may include photo-sensitive retinylidene proteins such as a channelrhodopsin. Channelrhodopsins may provide a controllable ion channel configured to actuate based on received light. Channelrhodopsin-1 (ChR1), Channelrhodopsin-2 (ChR2), or other variants are contemplated herein. Channelrhodopsins may provide sensitivity at various wavelengths of light. For example, the ChETA Channelrhodopsin may be opened by a blue light pulse and closed with a yellow light pulse. Such channelrhodopsins, other optically sensitive channel proteins, or other means for providing one or more neurons with sensitivity to optical stimuli could be provided to the second brain region via a genetic therapy, e.g., by infection of cells in the second brain region by a virus that includes genes coding for the channelrhodopsin or other proteins of other cell components. Other wavelength sensitive ion channels are possible.

Accordingly, stimulator 140 may include one or more light sources configured to actuate a channelrhodopsin or another type of light-sensitive ion channel. For example, stimulator 140 may include a blue laser diode coupled to a single-mode or multi-mode optical fiber, which may be delivered to the second brain region. In an example embodiment, the blue laser diode may provide 1.6 mW/μm^{2 at} 473 nm wavelength. The blue laser diode may be similar to that manufactured by CrystaLaser, CL473-025-O. Additionally or alternatively, stimulator 140 may include a yellow laser (593 nm wavelength, OEMLaser Systems, Inc., YL-593-00080-CWM-SD-05-LED-F). Stimulator 140 may also include various optical components, such as lenses, optical filters, and apertures, among oilier possibilities. As such, stimulator 140 may be configured to provide light at one or more wavelengths to the second brain region so as to indirectly provide an excitatory electrical signal to cells of the second brain region (via ion channels of the channelrhodopsin opening and closing).

In an example embodiment, waveform generator 130 may provide a gamma pulse or another type of waveform in response to a reference phase determined from an oscillatory signal detected using the sensor 110. For example, waveform generator 130 may provide to stimulator 140 three successive 5 ms square wave pulses with an inter-pulse interval of 15 ms. The onset of the gamma pulse may be triggered to coincide with an actual or predicted rising phase or other specified phase of the detected oscillatory signal.

In some embodiments, stimulator 140 may be configured to accept a digital signal input (e.g., the stimulator 140 may include one or more digital-to-analog converters). In such a scenario, waveform generator 130 may include a digital pattern generator (e.g., a lookup table, an arithmetic logic unit) configured to provide a digital representation of an electrical or optical stimuli to be provided to a given brain region. Other types of signaling between waveform generator 130 and stimulator 140 are contemplated.

Controller 150 may include a processor 152 and a memory 154, such as a non-transitory computer readable medium. Memory 154 may be configured to store instructions 156. Instructions 156 may be executed by processor 152 so as to carry out various operations described herein.

Processor 152 may include one or more general purpose processors—e.g., microprocessors—and/or one or more special purpose processors—e.g., image signal processors (ISPs), digital signal processors (DSPs), graphics processing units (GPUs), floating point units (FPUs), network processors, or application-specific integrated circuits (ASICs), Additionally or alternatively, the processor 152 may include at least one programmable in-circuit serial programming (ICSP) microcontroller. The memory 154 may include one or more volatile and/or non-volatile storage components, such as magnetic, optical, flash, or organic storage, and may be integrated in whole or in part with the processor 152. Memory 154 may include removable and/or non-removable components.

Processor 152 may be capable of executing program instructions (e.g., compiled or non-compiled program logic and/or machine code) stored in memory 154 to carry out the various functions described herein. Therefore, memory 154 may include a non-transitory computer-readable medium, having stored thereon program instructions that, upon execution by system 100, cause system 100 to carry out any of the methods, processes, or operations disclosed in this specification and/or the accompanying drawings. The execution of program instructions by processor 152 may result in processor 152 using data provided by various other elements of the system 100. Specifically, the controller 150 and the processor 152 may perform operations on based on information received from sensor 110 and/or data acquisition system 120. In an example embodiment, the controller 150 may include a distributed computing network and/or a cloud computing network.

The controller 150 may be configured to operate the elements of the system 100 to detect an oscillatory or otherwise repeating signal from a first brain region, to determine a reference phase or other timing information from the detected signal, and to provide at least one excitatory stimulus to a second brain region based on the determined reference phase. Such operation could be performed to provide repeated oscillatory stimuli to the second brain region that are synchronized with the timing of activity of the first brain region to induce a functional coupling between the first brain region and the second brain region (e.g., by potentiating or otherwise increasing the strength of stimulation received by cells of the second brain regions due to neurotransmitters released by efferent axons projecting from the first brain region to the second brain region) or to provide some other effect to treat a psychological condition or disorder.

In an example embodiment, controller 150 may be configured to receive, via the data acquisition system 120, information indicative of an oscillatory signal from a first brain region. Controller 150 may also be configured to determine, based on the received information, a reference phase of the oscillatory signal. Additionally, controller 150 may be configured to, in response to determining the reference phase, cause the waveform generator to generate at least one excitatory stimulus. For example, an onset of the generated at least one stimulus may be based on the reference phase of the oscillatory signal. Yet further, controller 150 may be configured to, in response to the generated excitatory stimulus, cause the stimulator to provide stimulation to a second brain region.

Additionally or alternatively, controller 150 may be configured to carry out some or all of the method steps or blocks described in relation to method 500.

Two or more of the elements of system 100 may be physically and/or communicatively coupled to one another via communication interface 160. Furthermore, communication interface 160 may allow system 100 to communicate, using analog or digital modulation, with other devices, access networks, and/or transport networks. Thus, communication interface 160 may facilitate circuit-switched and/or packet-switched communication, such as plain old telephone service (POTS) communication and/or Internet protocol (IP) or other packetized communication. For instance, communication interface 160 may include a chipset and antenna arranged for wireless communication with a radio access network or an access point. Also, communication interface 160 may take the form of or include a wireline interface, such as an Ethernet, Universal Serial Bus (USB), or High-Definition Multimedia Interface (HDMI) port. Communication interface 160 may also take the form of or include a wireless interface, such as a Wifi, BLUETOOTH®, global positioning system (GPS), or wide-area wireless interface (e.g., WiMAX or 3GPP Long-Term Evolution (LTE)). However, other forms of physical layer interfaces and other types of standard or proprietary communication protocols may be used over communication interface 160. Furthermore, communication interface 160 may comprise multiple physical communication interfaces (e.g., a Wifi interface, a BLUETOOTH® interface, and a wide-area wireless interface).

Power supply 170 may include one or more batteries. The batteries may include secondary (rechargeable) or primary (non-rechargeable) cells. Additionally or alternatively, at least some elements of system 100 may be powered by a conventional wall plug outlet (e.g., 120V, 60 Hz) or another type of energy source. In an example embodiment, power supply 170 includes an implantable battery that may be recharged via a wireless charging system.

A system as described herein for detecting an oscillatory signal from one or more brain regions and delivering excitatory stimuli to one or more further brain regions (e.g., providing the stimuli in sync with the phase or other properties of the detected signal to induce an increased degree of functional coupling between brain regions e.g., to treat the causes and/or symptoms of a psychological disorder or condition) could include a number of implanted and/or non-implanted elements or devices.

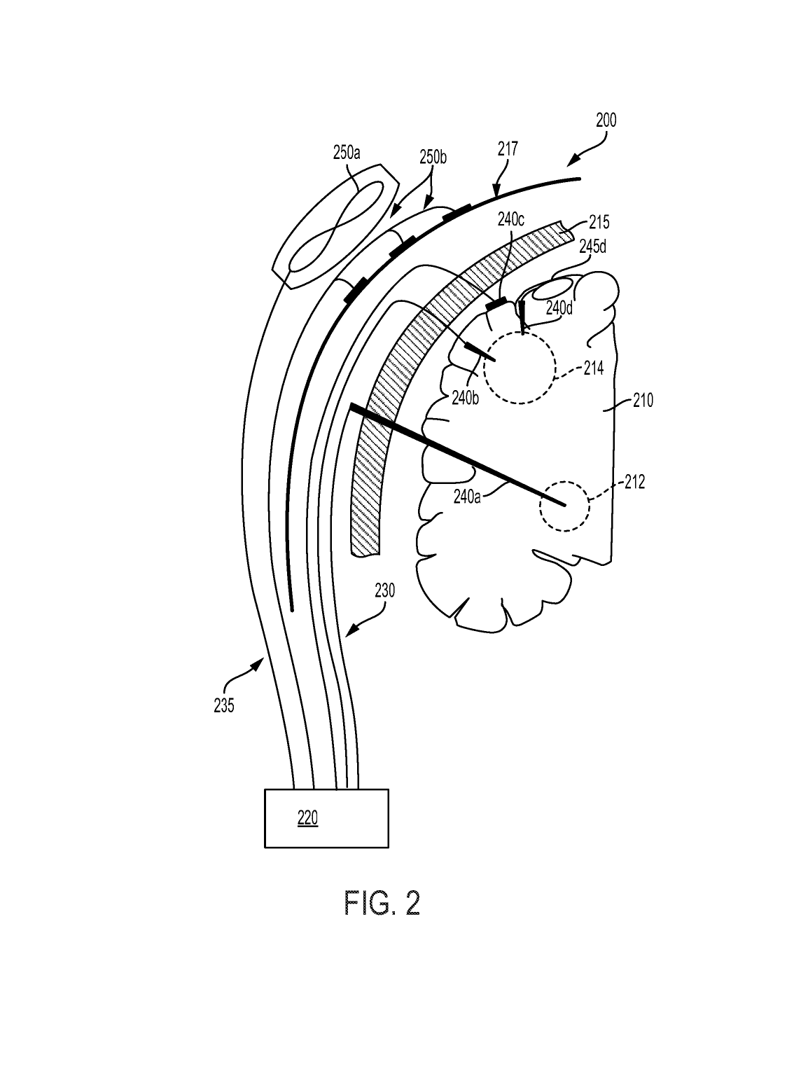

FIG. 2 illustrates elements of such a system. Some elements of the system 200 may be similar or identical to corresponding elements of the system 100 illustrated and described in relation to FIG. 1. Note that embodiments of systems as described herein may include more or fewer of the elements illustrated in FIG. 2. Elements of the system 200 are disposed on, within, or relative to a human body 200 that includes a scalp 217 disposed over a skull 215 that encloses a brain 210. The system 200 includes a controller 220 configured to operate the illustrated elements of the system 200. While the controller 220 is illustrated as a single device, in practice such a controller may comprise multiple separate elements (e.g., one or more implanted devices and/or one or more non-implanted devices, e.g., a user interface configured to communicate with other elements of the system 200 to effect operations of the system 200 in response to user inputs and/or to provide information or other indications to a user).

The system 200 includes non-implanted elements; specifically, a coil 250a disposed outside of and/or in contact with the scalp 217 and an array of surface electrodes 250b disposed on the scalp 217. The coil 250a could be configured and/or operated to provide transcranial magnetic stimulation to surface regions of the brain 210 (e.g., to the example surface region 214 of the brain). The array of surface electrodes 250b could be configured and/or operated to detect EEGs or other oscillatory signals from a target brain region, e.g., from the surface region 214.

The system 200 includes implanted elements. Some of the implanted elements are configured to access (e.g., to detect signals from, to provide excitatory stimuli to, or to otherwise interact with) the surface region 214 of the brain. Such elements include a surface electrode 240c, a first penetrating element 240b (e.g., an optical fiber, an electrode at the end of a rigid lead or other rigid element), and a second penetrating element 240d. Some of the implanted elements are configured to access a deep region 212 of the brain (e.g., a deep portion of temporal or other cortex, an aspect of the hippocampus, a nucleus of the striatum, limbic system, thalamus, tegmentum, hypothalamus, or some other deep structure(s)). Such elements include a cylindrical lead 240a that may include an optical fiber, one or more electrodes, wires, or other elements for detecting electrical, optical, or other signals from the deep region 212 and/or for providing electrical, optical, or other stimuli to the deep region 212.

As shown in FIG. 2, elements of the system 200 are connected to the controller 220 (e.g., to respective implanted and/or non-implanted aspects of the controller 220) via external cabling 235 or by implanted leads 230 disposed beneath the skin. Implanted leads 230 may be tunneled, beneath the skin, to a pocket beneath the pectoral muscle or to some other location at which elements of the controller 220 are implanted. Additionally or alternatively, the implanted leads 230 may pass through the skin to connect to a non-implanted aspect of the controller 220 and/or may lead to a connector or other elements of the system 200 that pass through the skin such that further cabling or other components may operably couple the leads 230 to a non-implanted aspect of the controller 220.

The second penetrating element 240d is coupled to an antenna coil 245d. The antenna coil 245d is configured to receive wireless energy and/or signals from another element of the system 200 (e.g., from the coil 250a). The second penetrating element 240d could be coupled to the antenna coil 245d via active electronic components (e.g., one or more microcontrollers or other integrated circuits). Such active electronic components could be configured to receive wireless energy, to transmit and/or receive wireless signals via the antenna coil 245d, and/or to operate the second penetrating element 240d to detect signals from the surface region 214 and/or to provide excitatory stimuli to the surface region 214. Alternatively, the second penetrating element 240d could be coupled to the antenna coil 245d via passive electronic components (e.g., one or more transformers, filters, capacitors, clamping diodes, inductors, resistors, or other passive components). Such passive components could be configured to transduce time-varying wireless fields received from an external source (e.g., form the coil 250a) into excitatory stimuli that could be provided, via an electrode, optical fiber, or other elements of the second penetrating element 240d, to the surface region 214. Note that such configurations could similarly be used to provide excitatory stimuli and/or to detect signals via the cylindrical lead 240a.

The cylindrical lead 240a and/or penetrating elements 240b, 240d may include an insulating cover that surrounds one or more wires that may be coupled to the electrodes of such elements 240a, 240b, 240d of the system 200 and/or that surrounds one or more optical fibers of such elements 240a, 240b, 240d of the system 200. In some embodiments, such elements 240a, 240b, 240d may include a biocompatible covering. In such a scenario, these elements 240a, 240b, 240d may be implantable.

The controller 220 may include various elements of system 100, such as data acquisition system 120, waveform generator 130, controller 150, power supply 170, and/or at least a portion of stimulator 140 (e.g., a laser light source). One or more aspects of the controller 220 may be configured to be implantable. However, in some embodiments, one or more aspects of the controller 220 need not be implantable. For example, the controller 220 may be worn or mounted externally.

One or more aspects of the controller 220 may include one or more controls, displays, outputs, or other elements of a user interface. The user interface may include a button, touch screen, touch pad, or another type of user interface device. The controller 220 may include at least one display. Such a display may include one or more indicator lights, a liquid crystal display, a graphical display, or another type of display. In some examples, a user interface of the controller 220 could be provided by a device or system in communication with the controller 220 (e.g., a smartphone, a computer or server in a physician's office). In some examples, a user interface of the controller 220 could include an implanted Hall effect sensor, magnetic reed switch, magnetometer, or other magnetic field sensing means configured to permit a user to provide inputs to the implanted aspect(s) of the controller 220 by passing magnetic object (e.g., a control pendant) over the implanted aspect of the controller 220 (e.g., to activate or deactivate the system 200, to control an intensity or other operational parameters of the system 200).

In an embodiment, the controller 220 may be controlled via such a user interface. Information indicative of a state of the system 200 and/or instructions for a use of the system 200 may be presented via a display of the user interface. For example, the display may display “SYSTEM NORMAL” when the system 200 is working normally. Furthermore, the display may display “PUSH BUTTON FOR THERAPY” to indicate that a patient may push a control (e.g., a button, an area of a touch-sensitive display) in order to initiate a therapeutic cycle or session, as described elsewhere herein. As an example, in the case where a patient may be prone to anxiety attacks, such a user interface may provide a way to controllably apply therapy when needed using the techniques described herein. Such a user interface may turn therapy on and/or off, provide adjustments for frequency, amplitude of therapy, etc.

As described elsewhere herein, a variety of systems and devices can be configured to detect oscillatory signals in a first brain region (or more than one first brain region) and to provide one or more pulses of excitatory stimulus in a second brain region (or more than one second brain region). Such stimulus can be provided to induce an increased functional coupling between the first and second brain regions (e.g., by providing the stimulus to the second brain region in sync with the timing of activity in the first brain region, as determined from the oscillatory signals detected from the first brain region) or to effect some other change(s) in the structure and/or function of the brain to address the causes and/or symptoms of a psychological condition or disorder.

Properties of such detection and stimulation (e.g., a property of the pulses of the provided stimulation, a timing of the pulses of stimulation relative to a reference phase or other features of the detected oscillatory signal) could be specified according to the psychological condition and/or disorder to be treated. In particular, the identity of the first and second brain regions (that is, the identity of the brain region to be stimulation and the identity of the further brain region whose detected oscillatory signals are to be used to determine the timing of the provided stimulation) could be specified based on the particular psychological condition or disorder to be treated.

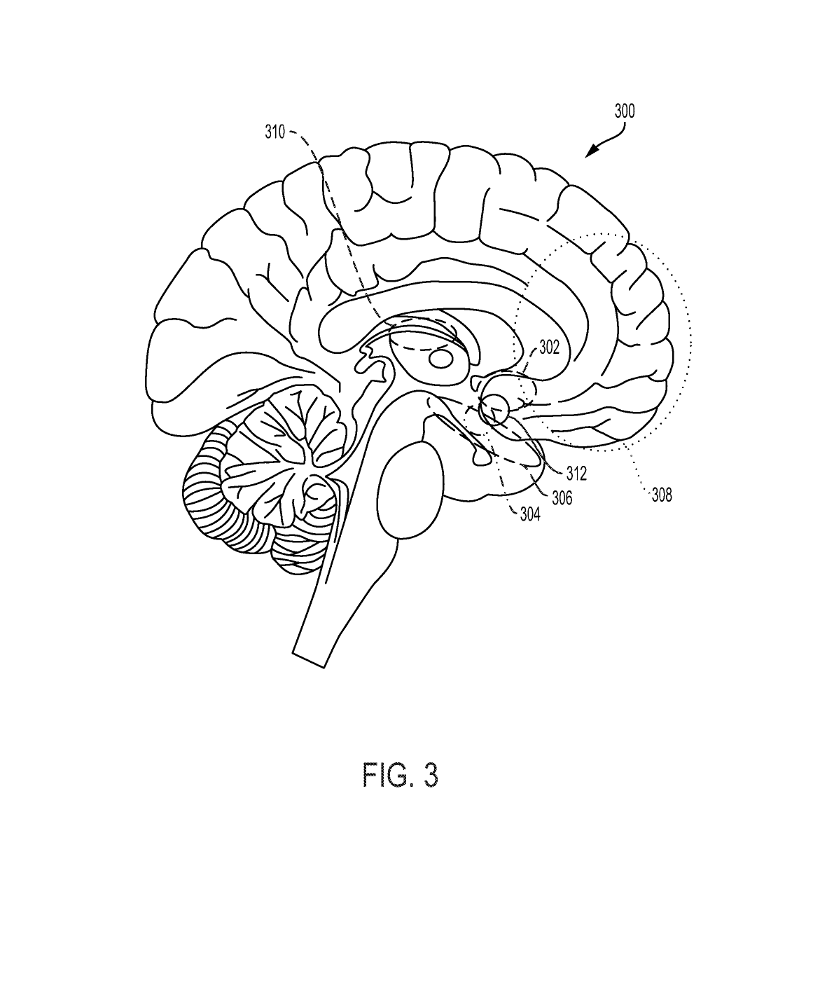

To illustrate the approximate relative locations of different brain regions that are relevant to the treatment of various psychological conditions or disorders using the methods and system described herein, FIG. 3 illustrates a human brain 300, according to an example embodiment. Note that the relative locations and extents of brain regions, as indicated in FIG. 3, are intended to provide examples only of the locations and extents of brain regions that could be monitored (e.g., by detecting an oscillatory electrical, optical or other signals) and/or stimulated. In practice, the locations and extents of such regions, within the context of the methods and devices described herein, may be determine experimentally, by consulting an atlas of the human brain, by performing some imaging or mapping of the anatomy, physiology, and/or function of a particular human brain, or by using some other method.

Further, while the illustration of FIG. 3 shows a sagittal image of the brain 300, some of the brain regions of interest are not located on a mid-sagittal surface of the brain 300. That is, some of the regions of interest may be located away from the mid-sagittal plane of the brain 300 and thus may be located into, or out of, the page, relative to the illustration of FIG. 3.

As noted above, in some example embodiments the specific sensing and stimulation regions of brain 300 may depend, at least in part, on the type of condition or disorder the therapy seeks to mitigate or relieve. For example, to alleviate bipolar disorder or other disorders of emotional or reward regulation (i.e. depression or addiction-related disorders), sensing may be performed in the subgenual cingulate cortex 302 or in prefrontal cortex 308 (e.g., ventral medial prefrontal cortex) and stimuli may be delivered to the nucleus accumbens 312 (or some other portion of the ventral striatum) or medial thalamus. In another embodiment, to alleviate post-traumatic stress disorder or other anxiety disorders, sensing may be performed in the subgenual cingulate cortex 302 (or in some other portion of prelimbic cortex) and stimuli may be delivered to the amygdala 304. In yet another embodiment, to alleviate symptoms of disorders related to memory or memory loss (e.g., schizophrenia, Alzheimer's disease), sensing may be performed in the hippocampus 306 (in particular, from the CAI region of hippocampus) and stimuli may be delivered to the prefrontal cortex 308. In a further embodiment, to alleviate disorders of social behavior like autism, schizophrenia, or depression, sensing may be performed in the medial dorsal (or dorsomedial) nucleus of the thalamus 310 and stimuli may be applied to the ventral striatum (e.g., to the nucleus accumbens 312).

Note that, while systems, devices, and methods are generally described herein as including detecting oscillatory signals from a first brain region and providing, based on the detected signals, excitatory stimuli to a second brain region, oscillatory signals may be detected from multiple brain regions and excitatory stimuli may be provided to multiple further brain regions based on the signals detected in one or more of the multiple brain regions. For example, oscillatory signals detected from multiple brain regions could be used to determine an aggregate reference phase, and stimuli could be provided to one or more brain regions based on the determined aggregate reference phase. In another embodiment, stimulation could he provided to the region from which oscillatory signals are received (e.g., to strengthen or weaken self-referent stimulation within the brain region). In yet another embodiment, multiple pairs of brain regions could simultaneously receive treatment from a device or system. For example, different oscillatory signals could be detected from first and second brain regions (e.g., left and right instances of a paired brain region, e.g., left and right prelimbic cortex) and different excitatory stimuli could be provided to third and fourth brain regions (e.g., left and right amygdala) based on respective detected oscillatory signals. Such pairings could be between regions on the same side of the brain (e.g., left prefrontal cortex and left amygdala) or between regions on opposite sides of the brain (e.g., left prefrontal cortex and right amygdala).

The phase and/or timing of stimulation provided to a first brain region, relative to a phase or other timing inflammation of oscillatory activity detected from a second brain region, could be specified according to an application. For example, the relative phase could be specified to strengthen a degree of functional connection between the first and second brain regions (e.g., to increase a magnitude of the response of the first brain region to synaptic inputs from the second brain region). Alternatively, the relative phase could be specified to weaken a degree of functional connection between the first and second brain regions (e.g., to decrease a magnitude of the response of the first brain region to synaptic inputs from the second brain region).

The relative phase between the detected oscillatory activity and the provided stimulation could be set based on a known or detected (e.g., based on electrophysiological measurements from a patient) relative phase or timing (e.g., a latency of synaptic transmission between the first and second brain regions) between activity in the two or more brain regions. Additionally or alternatively, the relative phase between the detected oscillatory activity and the provided stimulation could be set on an ongoing basis based on some detected signal, e.g., based on signals detected from the first and/or second brain region, signals related to an activity or emotional state (e.g., sleep, wake, exercise, mania, hypomania, depression) of a patient, signals related to a medication taken by a patient (e.g., an identity, dose, or timing of the medication). For example, the relative phase could be set to strengthen a connection between first and second brain regions during manic periods experienced by a patient and to weaken the connection between the first and second brain regions during depressive periods experienced by the patient.

It is understood that many other combinations of brain regions may be sensed and stimulated via techniques described herein. The possible brain regions for sensing and stimulation should. not be limited to those listed above and throughout this disclosure.

### III. Example Methods

As noted above, a variety of psychological conditions and/or disorders may be treated by detecting an oscillatory signal or other biosignal related to the activity of a first brain region and providing, to a second brain region, at least one excitatory stimulus having an onset that is based on a reference phase or other timing information about the activity in the first brain region determined based on the detected biosignal from the first brain region. Providing this excitatory stimulus could act to induce an increase the functional connectivity between the first and second brain regions (e.g., by increasing a sensitivity of cells in the second brain region to excitatory stimuli received from the first brain region via efferent axons of the first brain region) or to cause some other modification of the brain to effect a reduction or elimination of the causes and/or symptoms of a psychological condition or disorder.

As noted elsewhere herein, the detected oscillatory signal could be detected in a variety of different physical variables, related to a variety of different processes in a brain region that are related to the level of ongoing activity in the brain region over time. The detected signal could be an electrical field, current, or voltage, a magnetic field, a chemical concentration, an amount, wavelength, or other property of light emitted from the brain region (e.g., spontaneously, or in response to illumination, exposure to acoustical waves, or exposure to some other energy), a temperature, or some other physical variable. The signal could be detected from a single cell (e.g., the oscillatory signal could include a plurality of action potentials generated by a single cell and/or could include a firing rate over time of such a single cell), a number of cells in a particular volume of the brain region (e.g., the signal could be an LFP generated by the average activity of a population of cells), or a population of cells across an area or volume of the brain (e.g., the signal could be an electroencephalogram recorded from an area of multiple square centimeters).

Further, note that a detected oscillatory signal may be detected along with other signals (e.g., other oscillatory signals, at other frequencies, or noise signals). Thus, detected an oscillatory signals may include filtering a detected physical variable or other biosignal or performing some other processing or manipulation of a detected biosignal in order to detect an oscillatory signal of interest (e.g., an oscillatory signal corresponding to ongoing variations in the level of activity of a brain region over time).

In some examples, this could include filtering or otherwise processing a detected biosignal to generate an oscillatory signal having a frequency that is within a range of frequencies of interest, e.g., having a frequency between 2 Hertz and 7 Hertz or a frequency that is less than 20 Hertz. Detecting an oscillatory signal could include detecting which of a set of oscillatory signals within a range of frequencies of interest is the strongest (e.g., has the greatest amplitude, has the least distortion, has a waveform or shape that corresponds most to a waveform or shape of an oscillatory signal of interest) and filtering the detected biosignal to generate the strongest of the detected oscillatory signals. In some examples, a system or method as described herein could include determining that an oscillatory signal is present in a detected biosignal, and providing stimuli based on phase or other timing information determined from the biosignal only if the oscillatory signal is present. This could include determining that a ratio (or other comparison or function) between the power (or some other measure of magnitude) of the detected biosignal within a range of frequencies of interest (e.g., between 4 Hertz and 11 Hertz) and a range of noise frequencies or other unwanted frequencies (e.g., between 1 Hertz and 3 Hertz) is greater than some threshold determining that a signal-to-noise ratio of the detected biosignal is greater than some threshold).

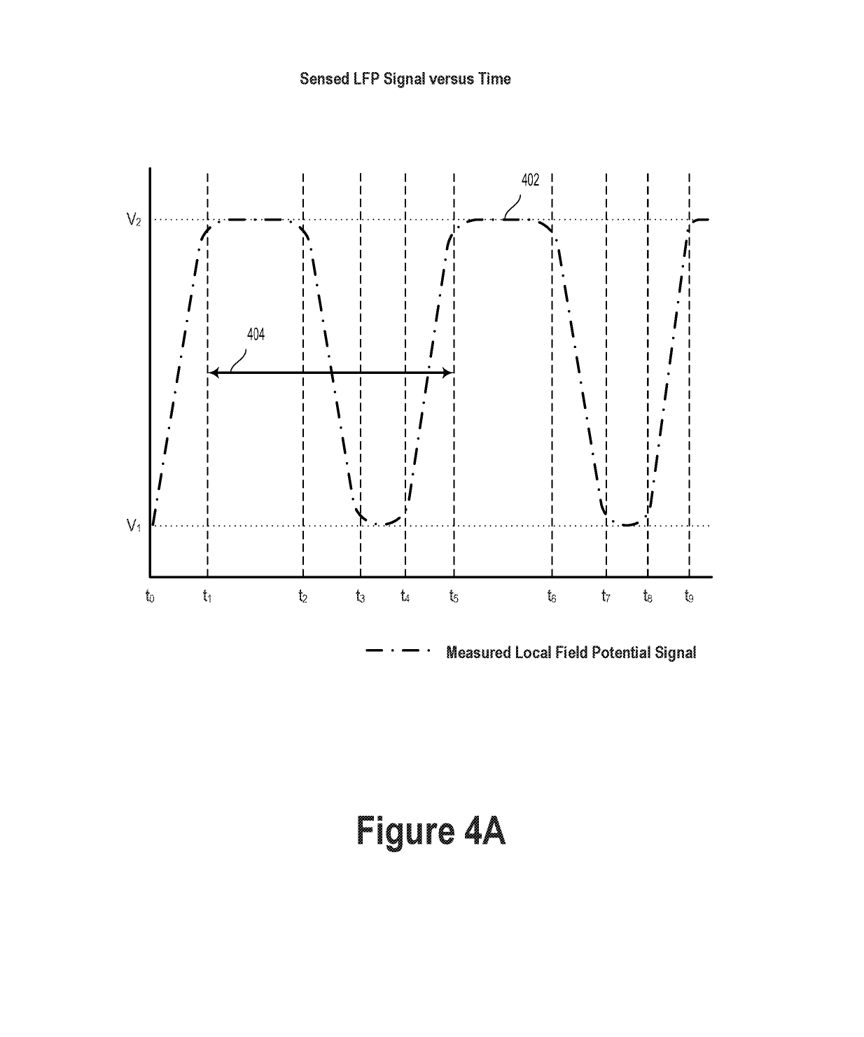

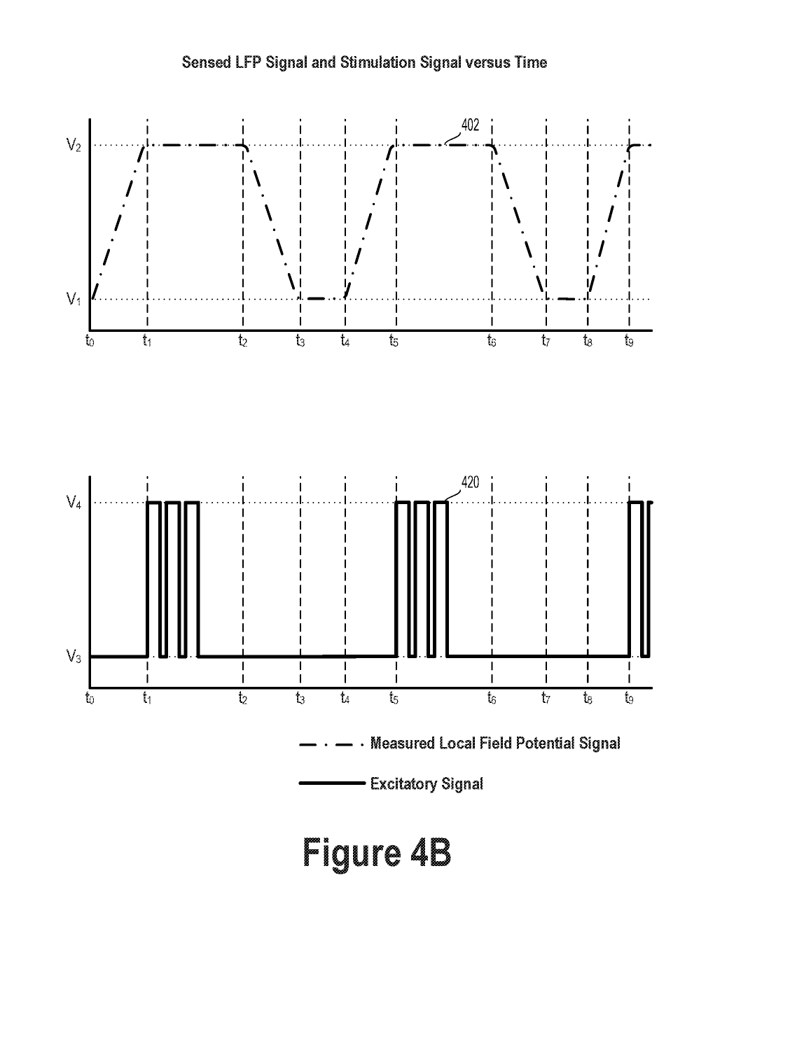

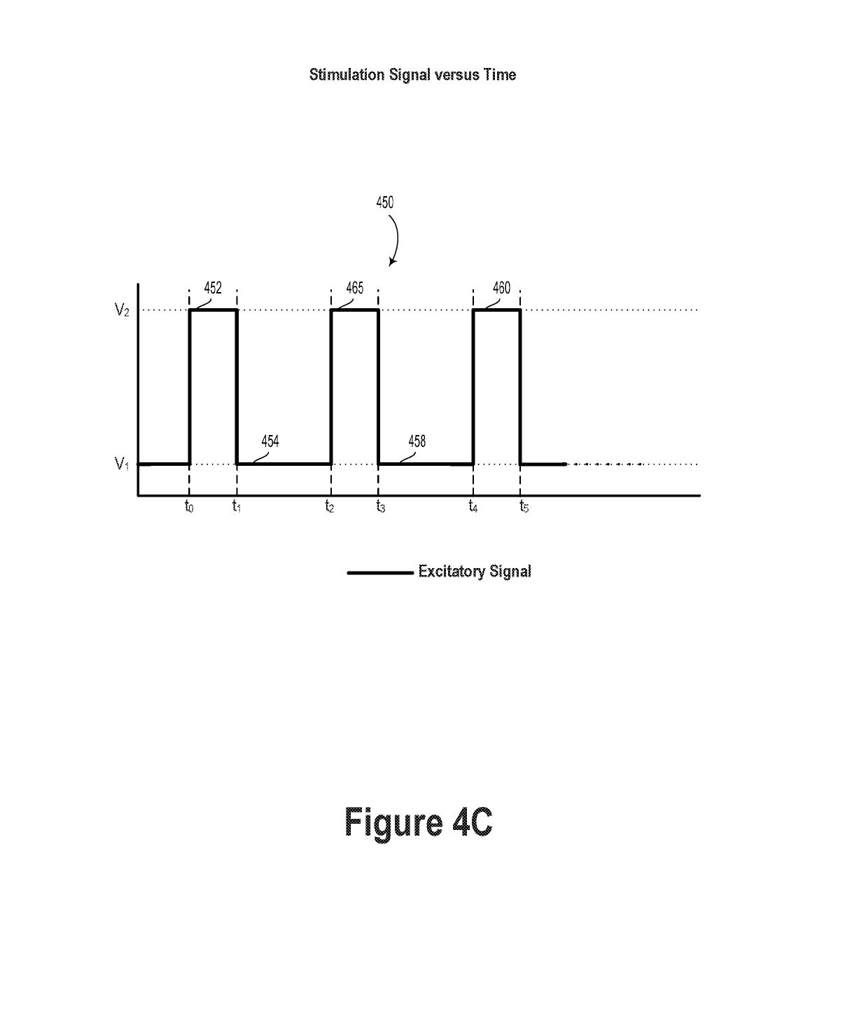

FIG. 4A illustrates an example oscillatory signal 402 (e.g., an LFP), according to an example embodiment. As described elsewhere herein, the signal 402 may be transduced by a sensor 110 and/or a data acquisition system 120. As illustrated, the signal may vary with respect to time according to a regular or semi-regular repeating pattern (e.g., as a sinusoid, a raised sinusoid, a sum of sinusoids, a sequence of pulses or other repeating or semi-repeating waveform patterns).

The period 404 of a single cycle of the signal 402 is illustrated in FIG. 4. The period of each cycle of a detected signal may vary slightly as the phase and/or frequency of the ongoing activity within a particular brain region (within a range of frequencies of interest) varies over time. As an example, a frequency of an LFP may be between 1-7 Hz, however other frequencies are possible. For example, for a 5 Hz oscillatory signal, a period 404 between t₅ and t₁ may be one-fifth of a second or 200 milliseconds.

A reference phase, a frequency, a modulation depth, or some other properties of the signal 400 could be determined and used to control properties or one or more excitatory stimuli provided to a brain region (e.g., a timing of the onset of a pulse of such an excitatory stimulus). One or more pulses of excitatory stimulus could be provided based on such determined information. For example, an excitatory stimulus could be provided at a specified time relative to a determined reference phase for each cycle of a detected oscillatory signal or for a subset of the cycles of the oscillatory signal (e.g., a repeating pattern of cycles, a randomly selected subset of cycles) and/or across a range of times relative to the determined reference phase. For example, excitatory stimulus could be provided to a second brain region, based on a reference phase determined from oscillatory signals detected form a first brain region, across a duration of time that is less than half of a period of oscillatory signals detected from the first brain region, or across a duration of time that is less than one quarter of a period of oscillatory signals detected from the first brain region.

The timing of onset of the stimulus relative to the determined reference phase could be static or could be based on some determination or method. For example, the relative timing could be altered to increase a detected coherence between oscillatory patterns within a first range of frequencies detected from a first brain region and oscillatory patterns within a second range of frequencies detected from a second brain region that is receiving the stimulus and/or the relative timing could be specified according to a determined latency between oscillatory signals detected from the first brain region and oscillatory signals detected from the second brain region.

A frequency, reference phase, duty cycle, harmonic distortion, depth of modulation, or some other properties of a given detected oscillatory signal may be determined based on various characteristics of the signal and/or using a variety of different methods. For example, the period may be measured based on subsequent detected falling edges t₂, t₆ or t₃, t₇ of the signal 402 or at a peak or trough of the signal 402. These edges, or other features of the signal 402, could be determined by comparing the signal 402 to a threshold, by applying a filter to the signal, by performing template matching to the signal, or by some other signal processing methods. A reference phase or other information could be determined for each of the cycles of the oscillatory signal (e.g., based on a detected rising edge of each cycle) and/or based on a number of cycles (e.g., based on the output of a phase-locked loop that receives the oscillatory signal as an input).

As an example of providing an excitatory stimulus having an onset or other timing properties that is based on a determined reference phase of a detected oscillatory signal, FIG. 4B illustrates an oscillatory signal 402 and an excitatory signal 420, according to an example embodiment. Controller 150 may cause waveform generator 130 to provide signal 420 directly to a stimulator (e.g., an implanted electrode), a light source, or some other stimulating means. In the case of the stimulus being an optical stimulus delivered via one or more light emitters (e.g., LEDs, lasers), signal 420 may have an amplitude v₄-v₃ of 5 volts or another enable/disable voltage for the light emitter. If the signal 420 is to be provided as electrical impulses, the voltage amplitude may vary based on a number of factors including the brain region of delivery and severity of underlying condition.

As shown in FIG. 4B, the excitatory stimulus provided for each of the cycles of the oscillatory signal 402 is substantially the same; that is, each of the stimuli includes the same number (three) of pulses (e.g., pulses of light, electrical current, electrical voltage, emitted neurotransmitter), each having substantially the same pulse widths, inter-pulse intervals, amplitudes, and waveforms. Further, the onset of each of the excitatory stimuli is substantially the same relative to the phase of a corresponding cycle of the oscillatory signal 402. However, one or more of these properties of the provided excitatory stimuli could vary between the stimuli and/or between pulses of an individual stimulus. For example, the inter-pulse interval of pulses of an excitatory stimulus could be specified based on a detected frequency of ongoing oscillatory activity in the brain region from which the oscillatory signal 402 was detected and/or in the brain region that is receiving the stimulus (e.g., to set the effective frequency of the pulses of the stimulus to correspond to a frequency, spectrum, or other property of the ongoing activity in one of both of the brain regions). The number of pulses, pulse widths, inter-pulse intervals, or other properties of each stimulus could be specified according to some consideration, e.g., to correspond to a period of a cycle of the detected oscillatory signal 402 such that the excitatory stimulus is provided across a specified range of phases relative to the cycle of the detected oscillatory signal 402. Such a range of phases could span less than 180 degrees (that is, have a duration less than half of the period of a cycle of the detected oscillatory signal 402), less than 90 degrees, or could span other range of phases that is sufficiently small to provide a modulated stimulus synchronized to the timing of repeated, oscillatory variations in the activity of the brain region from with the oscillatory signal 402 is detected.

In an example embodiment, each provided excitatory stimulus includes a gamma burst. A gamma burst includes two or more pulses of excitatory stimulus having pulse widths, waveforms, inter-pulse intervals, or other properties specified to induce activity within the stimulated brain region across a range of frequencies. The properties of such gamma bursts could be static (e.g., the inter-pulse intervals of such a gamma burst could be specified to correspond to an expected frequency of activity within a stimulated brain region) or could vary according to some factors or consideration (e.g., the inter-pulse intervals of such a gamma burst could be specified to correspond to a detected frequency of activity within a stimulated brain region). For example, the gamma burst may include 3 pulses with a rest period between each of the pulses. The pulse width could be 5 milliseconds and the inter-pulse interval may be 15 milliseconds. The pulse width and inter-pulse intervals may vary. An onset of one or more gamma bursts could be triggered by the detected oscillatory signal (e.g., a detected LFP) reaching a peak voltage. However, the gamma bursts may be triggered by other aspects or properties of a detected oscillatory signal (e.g., a rising edge, a falling edge, a trough, a phase determined using a phase-locked loop or other simal processing techniques, etc.).

FIG. 4C illustrates a segment of an excitatory signal 450, according to an example embodiment. Excitatory signal 450 includes three pulses 452, 456, and 460, which may make up a single gamma pulse or cycle. In an example embodiment, the three pulses 452, 456, and 460 are 5 milliseconds in duration and apply a 10-100 mV peak voltage (e.g., V₂-V₁). However, the duration of each pulse and the peak voltage (or other measure of the stimulus signal 450, e.g., emitted light intensity, applied electrical current, amount or rate of emitted neurotransmitter) may vary from pulse to pulse or from cycle to cycle. Between each pulse, there is an inter-pulse interval 454 and 458. In an example embodiment, the inter-pulse intervals may be 15 milliseconds; however each inter-pulse interval period may be different and inter-pulse intervals may vary from one to the next. Furthermore, while the total period of the illustrated example is 45 milliseconds (2 inter-pulse intervals of 15 milliseconds each and 3 pulses of 5 milliseconds each), shorter or longer periods are contemplated.

While FIG. 4C illustrates an excitatory signal 450 that includes a gamma pulse comprising three constituent pulses 452, 465, 460, more or fewer constituent pulses are possible. Furthermore, while square wave pulse shapes are illustrated, the pulse shape may vary. For example, the pulse shape may include, but is not limited to, a sine wave, a sawtooth, a ramp, or another arbitrary waveform shape.

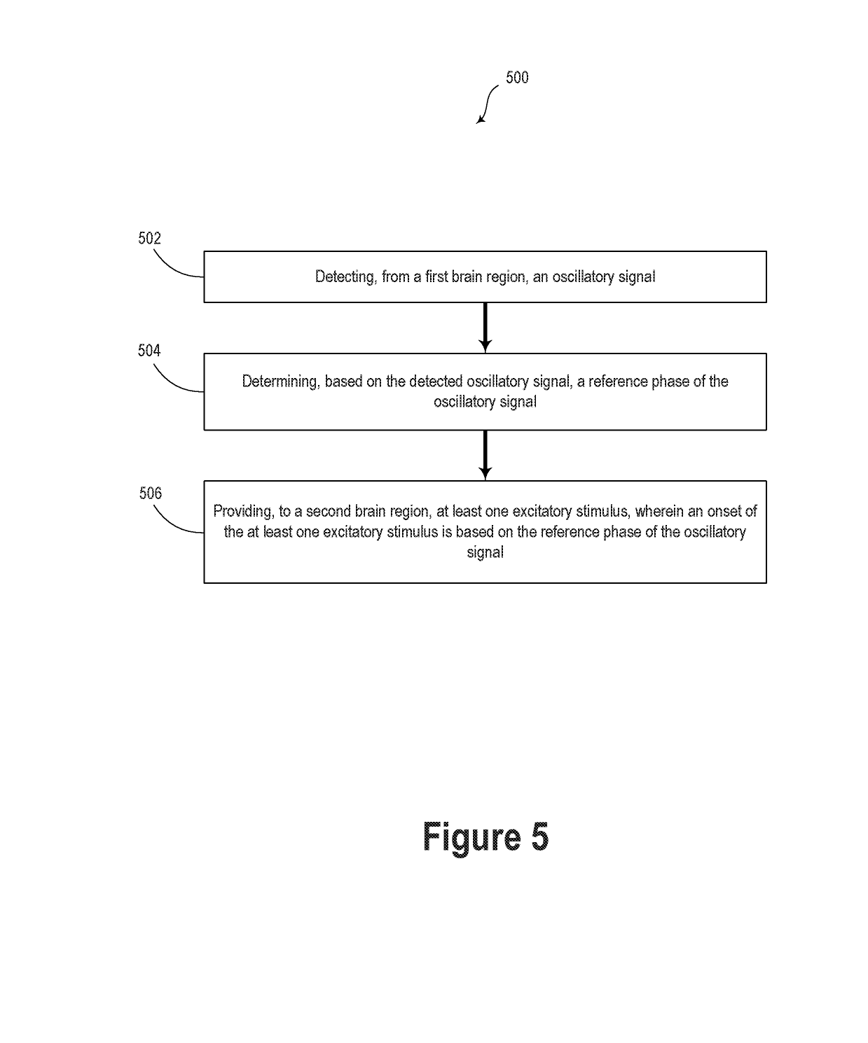

FIG. 5 illustrates steps of an example method 500 for providing a therapy to alleviate or otherwise treat the causes and/or symptoms of a psychological condition or disorder. The blocks of the method 500 may be performed by the systems and/or devices described herein. Blocks of method 500 may be conducted or carried out in a different order and any given block may be added, omitted, repeated, and/or looped.

The method 500 includes detecting, from a first brain region, an oscillatory signal 502. This could include detecting one or more of a variety of physical variables that vary over time in a manner related to the oscillatory signal. For example, the detected physical variable may relate to an electrical current, voltage, or field, a magnetic field, an intensity or other property of a light emitted from the brain region, a chemical concentration or concentration gradient, a temperature, or some other physical variable. Detecting the oscillatory signal could include emitting a light, an acoustical wave, or emitting some other energy to facilitate detection of a physical variable of interest, for example, emitting illumination to cause a calcium-sensitive dye or fluorophore to emit light having an intensity related to the concentration of intracellular calcium in one or more cells of the first brain region.

A detected physical variable could include a variety of signal components in addition or alternative to the oscillatory signal of interest, e.g., noise signals, motion artifacts, low-frequency offsets, or oscillatory signals that are not of interest (e.g., that have frequencies that are not within a range of frequencies of interest and/or that are within a range of frequencies of interest but that have an amplitude, level of distortion, waveform, or other properties that differ from those of an oscillatory signal of interest). In such examples, detecting an oscillatory signal 502 could include filtering or otherwise processing information indicative of the detected physical variable (e.g., an electrical signal, a plurality of digital codes) to generate information indicative of the oscillatory signal. As an example, the oscillatory signal 502 may be filtered using a phase-locked loop, such as by using a lock-in amplifier.

The method 500 also includes determining, based on the detected oscillatory signal, a reference phase of the oscillatory signal 504. This could include determining a timing of the oscillatory signal relative to a clock, generating a pulse or other signal based on a determined phase of the oscillatory signal (e.g., a timing pulse that could be provided to a waveform generator such that the waveform generator generates at least one excitatory stimulus having an onset based on the reference phase), or generating some other signals or information indicative of the determined reference phase. Determining a reference phase of the oscillatory signal 504 could include any of the methods described herein, e.g., determining the timing of a rising edge or other feature of the oscillatory signal, applying the oscillatory signal to a phase-locked loop or other signal-processing method to determine the phase or of the oscillatory signal, or performing some other filtering or signal processing techniques.

The method 500 further includes providing, to a second brain region, at least one excitatory stimulus, wherein an onset of the at least one excitatory stimulus is based on the reference phase of the oscillatory signal 506. This could include providing an electrical stimulus, an optical stimulus, a chemical stimulus, or some other excitatory stimulus to the second brain region. The provided stimulus could include a gamma pulse or some other type of excitatory stimulus having an onset determined based on the determined reference phase. An excitatory stimulus could be provided for each cycle of the oscillatory signal, or some subset of the cycles of the oscillatory signal. Each provided excitatory stimulus could be substantially the same or could vary (e.g., such that an effective frequency of pulses of each excitatory stimulus corresponds to a detected frequency of activity in the second brain rethon).

The method 500 could include additional or alternative steps. For example, the method 500 could include receiving one or more commands for a patient and/or clinician. Steps of the method (e.g., 506) could be performed in response to receiving certain commands (e.g., a command to begin providing a therapy) and/or operational parameters of the performance of the method 500 could be set based on received commands (e.g., an amplitude, frequency, timing relative to a determined reference phase, or other properties of the provided stimulus could be set according to received user inputs). The method could include determining whether an oscillatory signal of interest is present in a detected biosignal (e.g., in a detected LFP) using a variety of methods (e.g., by determining a signal-to-noise ratio of the detected biosignal and comparing the determined signal-to-noise ratio to a threshold) and providing stimulus to the second brain region 506, among other steps of the method 500, may be performed based on such a determination (e.g., such that substantially randomly-timed stimuli are not provided to the second brain region during periods of time when the oscillatory signal cannot be detected).

### IV. Example Therapeutic Methods

The methods, systems, and devices described herein could be used to provide a therapy to a person, e.g., to alleviate the symptoms and/or causes of one or more psychological conditions or disorders. For example, the methods, systems, and devices described herein could be used to treat bipolar disorder or other mood disorders, post-traumatic stress disorder or other anxiety disorders, schizophrenia and/or memory loss, autism, or other psychological conditions or disorders.

Such therapies could be provided in response to a diagnosis that a patient is experiencing one or more relevant psychological disorders or conditions. Further, such therapies could be provided following the application of other therapies having incomplete success or effectively in treating the causes, symptoms, or other aspects of a diagnosed psychological condition. For example, if providing the therapy is predicated by the implantation of one or more devices or electrodes (e.g., a cylindrical lead to access deep structures within the brain and/or penetrating or surface elements to access surface structures of the brain, along with any associated leads, implanted controllers, transcutaneous connectors, or other implanted elements), less invasive therapies (e.g., talk therapy, pharmaceutical interventions) may be pursued before applying therapies as described herein.

Providing such a therapy could include detecting an oscillatory signal in a first brain region and providing, to a second region, one or more excitatory stimuli having an onset time or other timing properties determined relative to a determined phase of the detected oscillatory signal. Oscillatory signals could be detected from a number of different brain regions and/or excitatory stimuli based on such detected signals could be provided to multiple different brain regions.

The therapy could be provided chronically (e.g., continuously or as a number of discrete therapies over months or years) or as an acute therapy (e.g., one or more discrete sessions). An acute therapy, or discrete instances of a chronic therapy, could be provided by devices that are present in a physician's office or hospital. For example, oscillatory signals could be detected using an array of EEG electrodes, a magnetoencephalogram, a magnetic resonance imager, or other devices or systems. Conversely, excitatory stimuli could he provided by transcranial magnetic stimulation. Additionally or alternatively, aspects of an acute therapy (e.g., detection of an oscillatory signal and/or providing excitatory stimuli) could be provided by implanted devices that may be removed and/or disabled following completion of the therapy. An acute therapy could be provided for a specified period of time, number of sessions, or amount of deliver stimulation. Additionally or alternatively, an acute therapy could be provided until to condition is detected, e.g., until a coherence between first and second brain regions increases above a threshold amount, until a specified degree of alleviation of signs or symptoms of a psychological condition or disorder has been observed, or until some other condition has been met.

Providing such a therapy could include implanting one or more devices. For example, providing such a therapy could include implantation of sub-dural or supra-dural electrodes, surface electrodes, penetrating electrodes or optical fibers, leads, controllers, or other implanted elements of a device or system. Some of these elements could be implanted stereotaxically, e.g., to facilitate the positioning of stimulating means and/or signal-detecting means in a target brain region. Implantation of such elements could include performance of test stimulation and/or recordings to verify the accurate placement of such elements intra-operatively. In examples wherein oscillatory signals are to be detected optically and/or wherein stimulation is to be provided optically, a genetic therapy or other substance could be introduced to one or more brain regions (e.g., to infect cells of a brain region with a gene to induce production of a calcium-dependent dye or fluorophore or a channelrhodopsin or other optically-sensitive substance)

Such a therapy could include determining settings for provision of the therapy. Such settings could include amplitudes, dosages, pulse waveforms, frequencies, or other properties of provided stimulus. Such settings could include determining a relative timing between a determined reference phase of a detected oscillatory signal and the onset of a provided stimulus (e.g., to maximize a positive effect of the stimulus on one or more brain regions). The determination of such settings could be automated based on one or more detected signals applied to an algorithm or some other automated method of parameter determination) and/or manual (e.g., a clinician could manually vary stimulation parameters until a desired effect is achieved).

Providing such a therapy could include providing adjunct therapies in combination with the provision of excitatory stimuli in time with detected oscillatory brain signals. For example, pharmaceuticals could be provided in addition to such stimulation. The amount of such pharmaceuticals provided to a patient could be decreased as the effects of the stimulation increase.

### V. Experimental Results

In vivo neurophysiological recordings were performed concurrently across eight distinct cortical and limbic brain regions in mice subjected to a repeated tail suspension test (TST). It was found that repeated exposure to the TST reduced escape behavior and altered neural activity in two distinct network nodes: one centered in infralimbic cortex and the other in the ventral tegmental area (VTA). The infralimbic cortex (IL) in rats may be homologous to the subgenual cingulate cortex or other regions of the prelimbic cortex in humans. These mesoscale nodal adaptations were accompanied by changes in the cellular response profiles in IL, but not VTA. Furthermore, these mesoscale changes did not occur in the VTA-node of a genetic mouse line that exhibits resilience to multiple behavioral stressors including the TST, and closed-loop optogenetic stimulation of IL-dependent circuitry in normal mice showed that their IL-network adaptations were compensatory, not primary. Finally, direct stimulation of dorsal raphe serotonin neurons using designer receptors exclusively activated by designer drugs (DREADDs) induced a network state in the VTA-node that directly opposed the maladaptive network changes that followed repeat TST exposure.

Taken together, these findings demonstrate that neural adaptations in a mesoscale VTA-network node play a central role in mediating the pathological behavioral changes that occur in responses to challenging experiences. These changes, and the challenging experiences that cause them, may be considered analogous to post-traumatic stress disorder, generalized anxiety disorder, or other psychological disorders experienced by humans in response to traumatic or otherwise challenging experiences. Furthermore, we show that the two major brain structures (VTA and IL) proposed to mediate stress induced behavioral changes lie within a single mesoscale network.

Experiments described herein relate to systems and methods involving in vivo neurophysiological recordings of action potentials and local field potentials (LFPs) from eight cortical and limbic brain regions that define networks for emotional behavior. The recordings were performed while animals were subjected to a tail suspension test (TST). The TST is a classic assay used to probe the impact of pharmacotherapeutics on the behavioral response of mice to a challenging experience. In this assay, mice are subjected to an inescapable stressor. The inescapable stressor includes being suspended upside-down by their tail. The test induces a robust stress response, and the time animals spend immobile relative to the time they spend engaging in escape actions is interpreted as an indicator of their behavioral response to the challenge. TST behavior is sensitive to the same classes of pharmacological agents used to treat stress induced behavioral disorders in patients (e.g., antidepressants). Thus, the TST can be used to probe the function of mesoscale neural networks during a challenging experience.

Experiments were performed to determine the strength of the responses in each brain area and the functional connectivity between areas during an initial TST session in naïve mice. Experiments were then repeated on the day subsequent to the initial TST session. This approach diminished behavioral responses of the mice to the challenging experience, as indicated by decreased motor activity of the mice. Furthermore, repeat TST exposure induced adaptations in a multi-area mesoscale network with two nodes: one centered in IL and the other centered in VTA. A novel closed-loop optogenetic stimulation system was then applied to reveal that the network adaptations involving the IL were compensatory, while DREADD stimulation experiments showed that the VTA-node adaptation was primary. Thus, these findings linked specific mesoscale neural network adaptations with antidepressant responses and the ememence of behavioral pathology.

Abbreviations may be used throughout this disclosure to indicate regions of the mouse and/or human brain. Unless indicated otherwise, regions of the brain are abbreviated as follows:

Nucleus Accumbens: NAc; may refer to nucleus accumbens as a whole, or specifically to the shell or core of the nucleus accumbens.

Prelimbic cortex: PrL.

Infralimbic Cortex: IL.

Thalamus: Thal.

Medial dorsal nucleus of thalamus: MD-Thal.

Ventral Tegmental Area: VTA.

Amygdala: AMY.

Hippocampus: Hip.

Dorsal hippocampus: D-Hip.

A. Actions Motivated by Behavioral Challenge Recruit Distributed Neural Architecture.

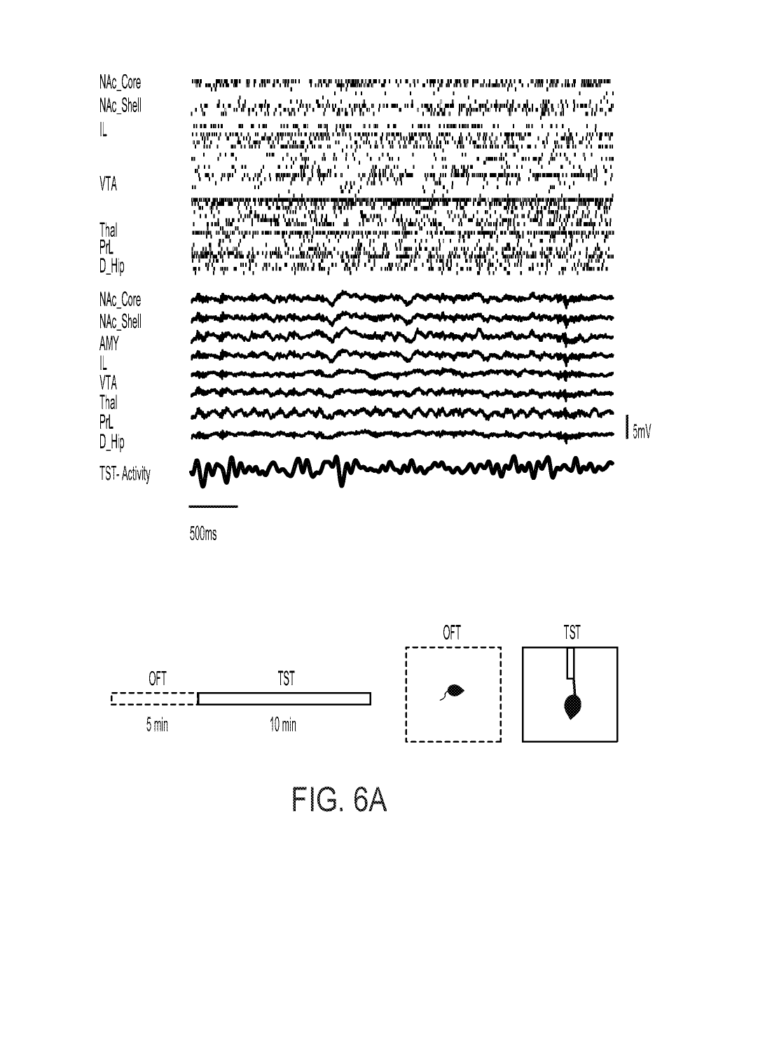

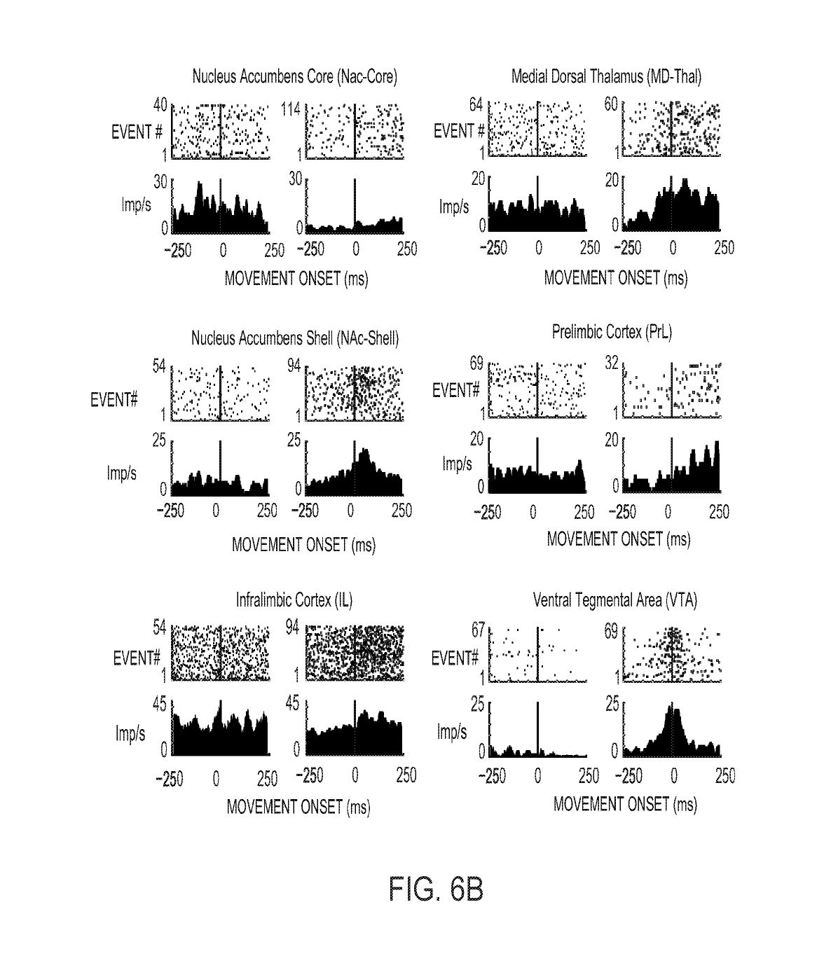

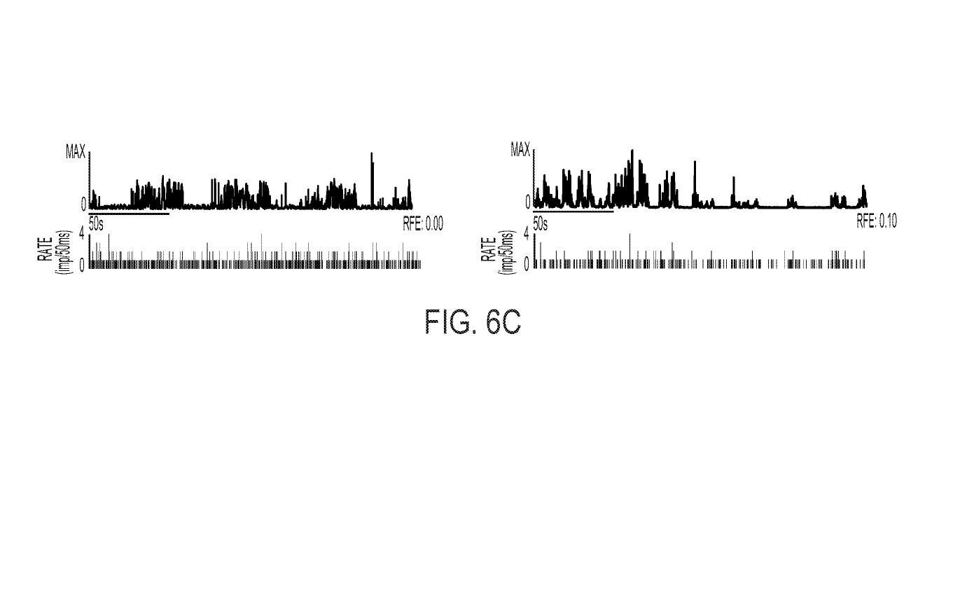

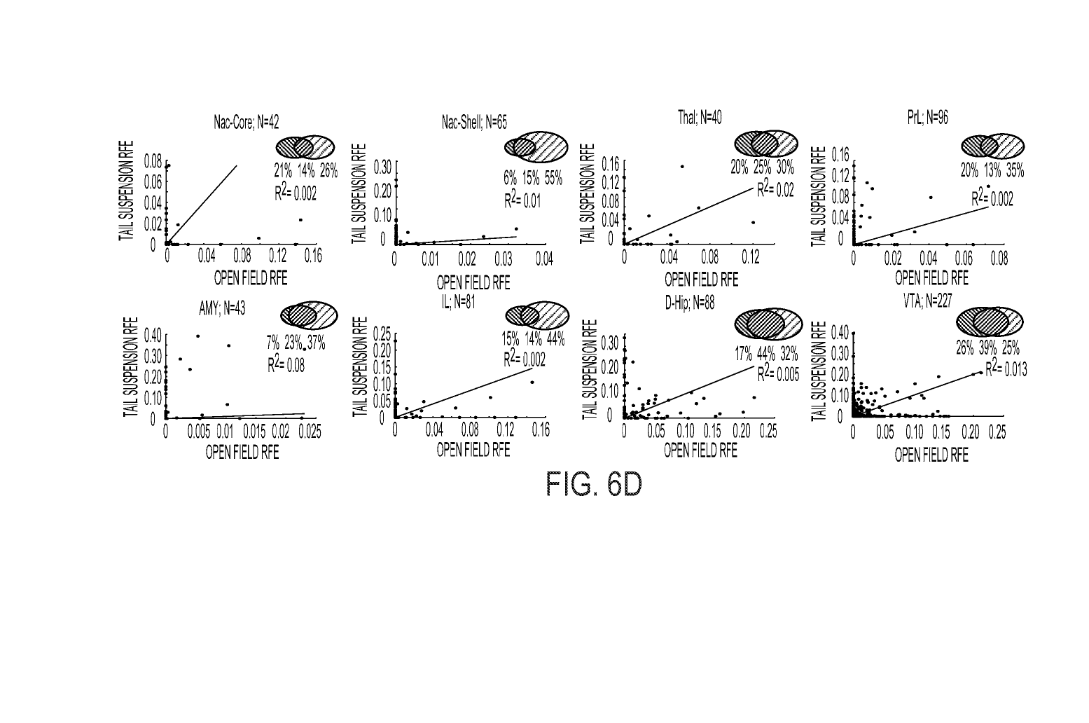

Mice were subjected to an open field test followed immediately by the TST. The spatial location of each mouse was recorded continuously during the open field test, and behavioral activity was measured continuously during the TST. These behavioral measures were used as indicators of instantaneous movement. Neurons were definitively identified from each monitored cortical and limbic brain area that signaled movement during the TST, open field, or both tests. FIGS. 6A, 6B, and 6C illustrate some of the results of these experiments. FIG. 6A includes, at the top, a raster plot showing unit and local field potential (LFP) activity acquired concurrently from cortical and limbic brain areas during the tail suspension test (TST). The bottom of FIG. 6A includes a schematic of the behavioral recording sessions and a timeline of the phases of the experiment. FIG. 6B includes raster plots and peri-event time histograms of example neuronal firing from a variety of different brain regions time-locked to movement onset in the open field test (OFT; the left plots for each brain region) and TST (the right plots for each brain region).

To quantify these spike-behavioral relationships, a linear relationship was used to model behavioral activity within each behavior test session based on the spiking of each neuron. This is shown by example in FIG. 6C, which shows firing rate histograms of a NAc-Shell neuron (bottom) of a mouse relative to the movement activity trace of the mouse (top) during the OFT (left) and TST (right). This linear relationship model yielded an error metric based on a determined reduction in fractional error (RFE). The RFE provides a measure of the amount of behavioral variance explained by the firing of each individual neuron. The linear model predicts an activity trace for each neuron that models movement in the a window that extends form 1.2 seconds before spiking to 1.2 seconds after spiking.

Substantially no correlation was detected between the extents to which neurons signaled movement across the two behavioral paradigms in any of the brain regions recorded. This is illustrated in FIG. 6D, which includes plots showing the relationship between spiking and behavioral activity for each neuron measured using an error metric based on the reduction in fractional error (RFE). The black line indicates the unity line along which a neuron signals action equally in both tasks. Venn diagrams quantify the portion of neurons in each brain area that signal action during each behavioral test (RFE>0). TST-RFE and OFT-RFE for each brain area was compared using Spearman rank correlation (R<0.1 for all comparisons).

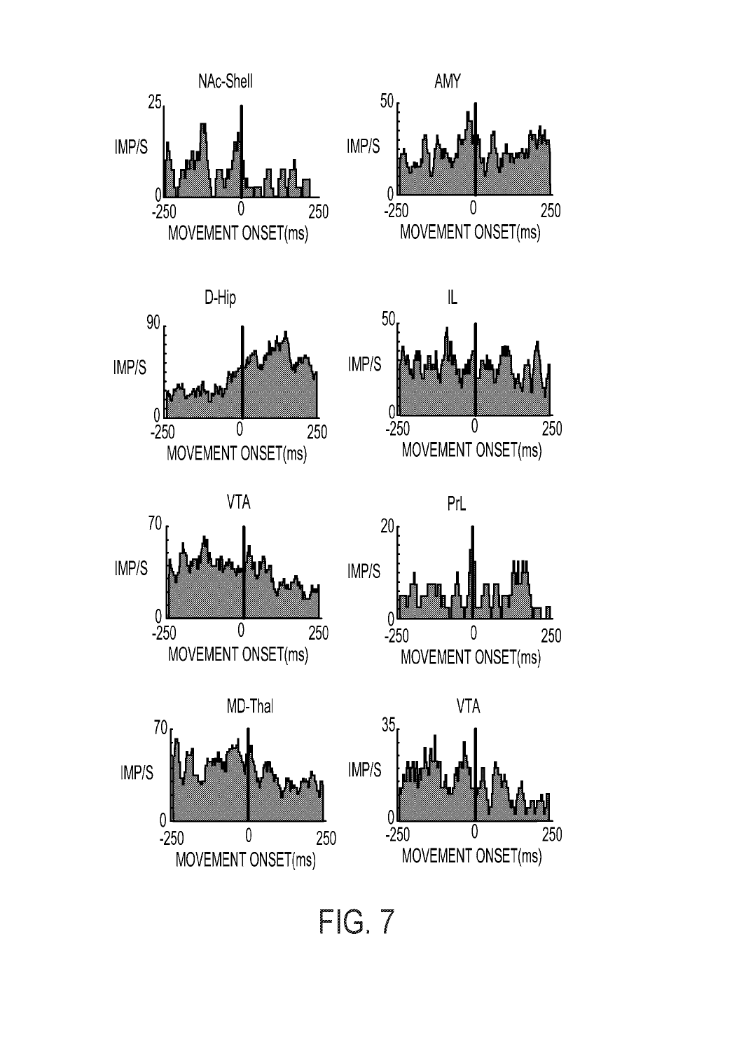

Thus, neurons that signaled movement during one assay did not necessarily signal movement in the other, demonstrating that unit responses during the TST did not simply reflect general movement or global movement artifacts. This was further supported by the activity profiles for multiple neurons that showed increased firing rates prior to the onset of movement in the TST. Examples of such activity profiles are illustrated in FIG. 7, which includes peri-event time histograms from a variety of different brain regions showing unit spiking relative to movement onset during the tail suspension test. Note that multiple neurons show peak firing prior to movement onset. Others show rate increases prior to movement onset, or no event related firing. This demonstrates that unit responses during the tail suspension test were not simply due to movement artifact.

Taken together, neurons within all of the recorded cortical and limbic regions exhibited firing patterns that were linked to the specific escape behaviors motivated by the TST assay. This is consistent with prior reports of prelimbic cortex and nucleus accumbens responses during escape behaviors in the forced swim test, another stress-related behavioral assay.

B. Selective Adaptation in Escape Architecture Following Repeated Behavioral Challenge

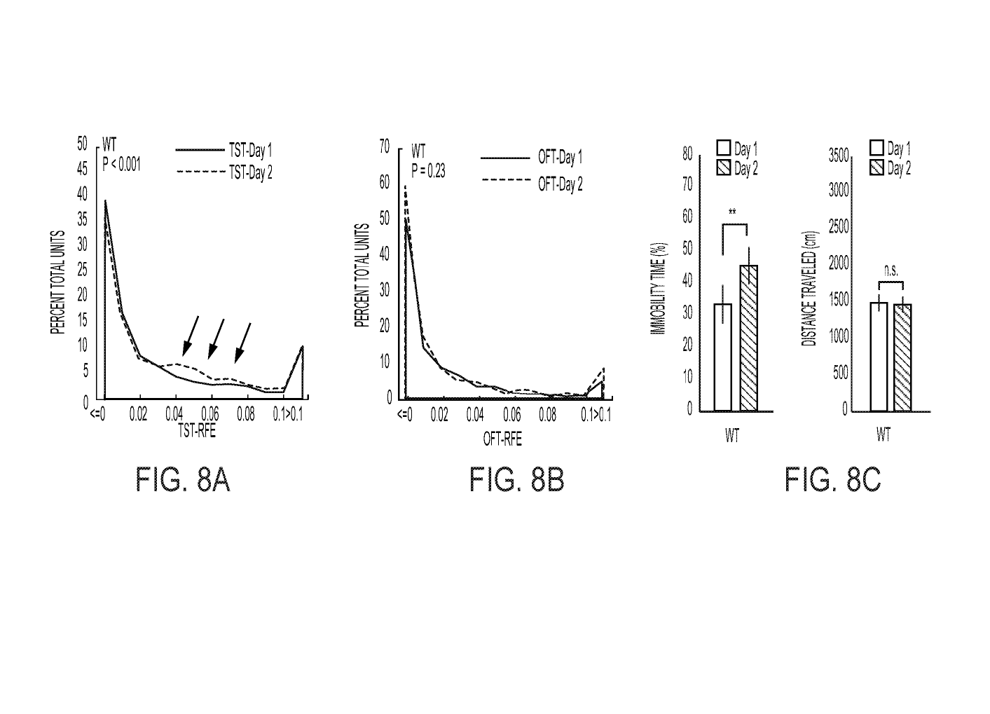

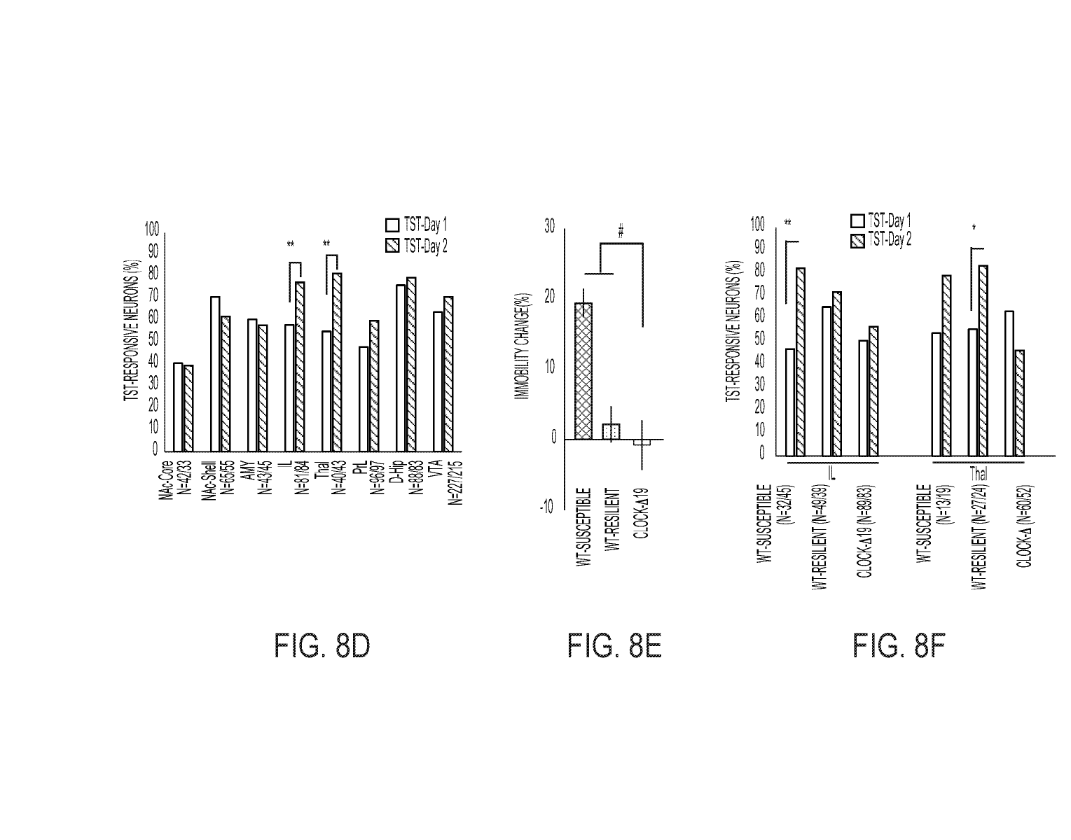

Experiments were performed to determine whether repeated exposure to the behavioral challenge induced adaptations in the spike-behavioral response profiles described above. An increase in TST-related neural signaling was detected on the second day of exposure to the TST. That is, the population of neurons exhibited an increased ability to predict behavioral responses on the repeat testing day, assessed as an increase in the RFE provided by the linear model for each neuron (P<0.001, using Kolmogorov-Smirnov test; N=647 and 620 neurons during sessions on the first and seconds days, respectively). This is illustrated in FIG. 8A, which shows histograms of test related neuronal firing during the TST as applied on the first and second days of the experiment (histogram points indicated by arrows exhibited P<0.05 using Kolmogorov-Smirnov test).

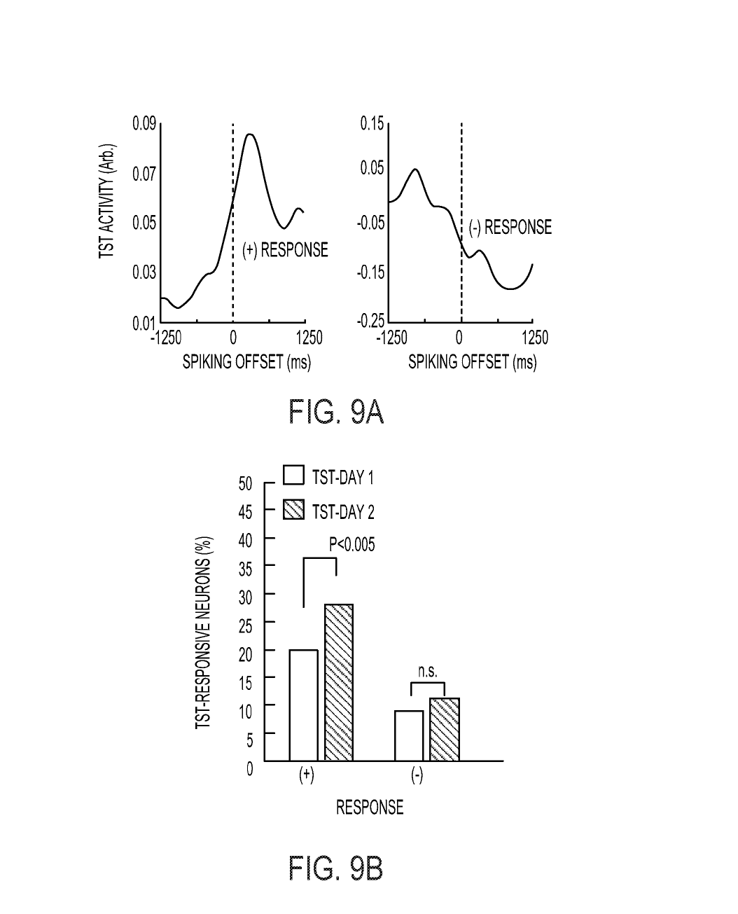

No difference was observed in open field movement-related signaling (P=0.23, using the Kolmogorov-Smirnov test. This is illustrated in FIG. 8B, which shows histograms of test related neuronal firing during the OFT as applied on the first and second days of the experiment. This increase in TST-related neural signaling reflected a decrease in the efficiency of neural circuits that transition the animal from immobility to mobile periods. This is illustrated by FIGS. 9A and 9B. FIG. 9A illustrates TST-activity within a window from 1.25 second before to 1.25 after neural spiking a predicted by two example spike-activity models. FIG. 9B illustrates that a higher portion of positive slope ((+) RESPONSE) neurons was observed during repeat testing. This increase in TST-related neural signaling indicates a decrease in the efficiency of neural circuits that transition the animal from immobility to mobile periods.

Supporting this notion, animals exhibited higher immobility during the second testing session. This is illustrated in FIG. 8C (p=0.002 using paired t-test). No difference in distance traveled was observed in the open field on the second day, demonstrating that the behavioral adaptation, like the neural adaptation, was specific to the challenging experience. This is illustrated in FIG. 8C (p=0.74 using paired t-test).

Experiments were performed to determine the brain region-specific adaptations responsible for the altered cellular response profiles. Only IL and Thal showed significant increases in TST-responsive neurons. This is illustrated in FIG. 8D, which shows region-specific expansion of TST-related movement function (double-starred pairs, which include IL and Thal, differ at P<0.05 using G-test with Williams correction). Experiments were then performed to determine whether these IL and/or Thal neural adaptations were a hallmark feature linked to the behavioral decline displayed by animals.

These experiments were performed after dividing a group of wild-type experimental animals into two groups based on each animal's behavioral adaptation across the two TST sessions. The groups included a WT-susceptible group, which contained mice that showed increases in immobility during the second TST session. The groups also included a WT-resilient group, which contained mice that did not show increases in immobility during the second TST session. This increase is illustrated by FIG. 8E; the WT-susceptible group and WT-resilient group each included 7 animals. The difference between the immobility change for each of these two groups was different, with a P=7.7×10−5 using two-tailed Student's t-test within subjects.

Experiments were also performed using a genetic mouse line (Clock-Δ19 mice) that has been previously shown to exhibit resilience to multiple distinct assays of behavioral challenge including the forced swim test and a learned helplessness test. The Clock-Δ19 mice were resilient to the adaptation induced by repeated TST exposure relative to the full group of normal animals (P=0.017; F1, 25=6.51 for session×genotype interaction using two-way ANOVA, P=0.83 for post-hoc testing using a paired t-test within genotype). This increase is also illustrated by FIG. 8E. No differences in TST immobility were observed across genotype within the first testing session (P=0.75 for post-hoc testing across genotype using unpaired t-test; data not shown),

The increase in IL TST-responsive neurons was only observed in the subset of WT animals that showed the greatest increase in immobility across testing sessions (i.e., WT-susceptible; P<0.05 using G-test with Williams' correction; G-Value=10.5, df=2), while Thal tended to show this increase irrespective of the extent to which animals showed behavior changes across TST testing sessions (i.e., WT-Resilient; P=0.03 using G-test with Williams' correction; G-Value=4.6; df=2; WT-Susceptible: P=0.15 using; G-Value=2.1; df=2). This is illustrated in FIG. 8F; double-starred difference different at P<0.01 and single-starred difference different at P<0.05 using G-test of independence across session with Williams correction. No changes in IL or Thal TST-activity were observed in the Clock-Δ19 mice. Thus, the functional expansion of IL that occurred after behavioral challenge was directly linked to the susceptibility of animals to the prior TST exposure, while the thalamic expansion tended to occur in all wild-type (WT) animals.

C. Repeated Exposure to Behavior Challenge Induces Adaption in IL-Thal Circuitry

The infralimbic cortex-medial dorsal thalamic (IL-Thal) circuit includes unidirectional monosynaptic efferents from IL to Thal. Experiments were performed to determine whether adaptations in this circuit accompanied the adaptations we at the cellular level. These experiments included testing whether repeated exposure to behavioral challenge also induces changes in LFP activity of one or both of these brain regions. Cross-frequency phase coupling (CFPC) analysis was used to analyze data from these experiments. CFPC has been shown to signal emotional responses across cortico-limbic circuitry. It was determined that the phase of 3-7 Hz activity in IL was coupled to the amplitude of low-gamma activity (30-70 Hz) in Thal. Determined CFPC between these structures increased across testing sessions (P=0.0017 using sign-rank test), similarly to the neuronal and behavioral changes shown above.

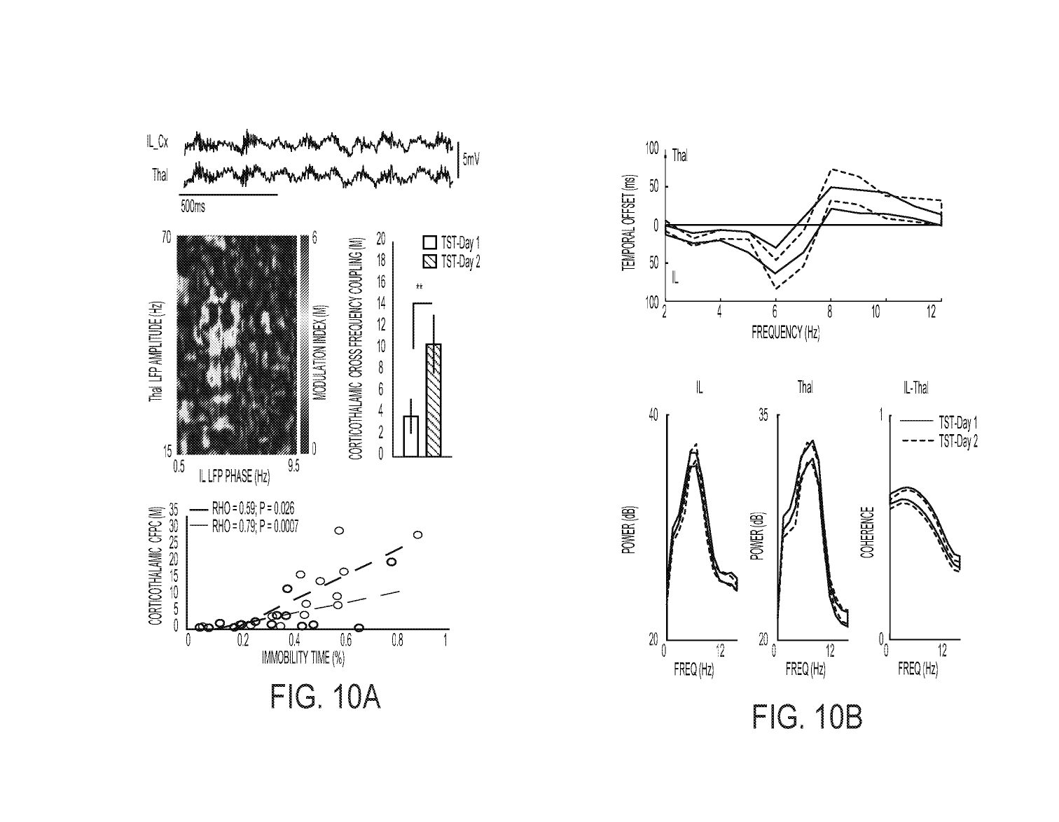

Experimental results relating to CFPC are illustrated in FIG. 10A. The top of FIG. 10A shows example IL and Thal LFP traces. CFPC was calculated using the modulation index of the recorded IL and Thal LFP signals. The coupling between IL 3-7 Hz oscillations and Thal 30-70 Hz oscillations increased across testing sessions (P<0.05 using sign-rank test; shown in the middle of FIG. 10A). The bottom of FIG. 10A shows that IL-Thal CFPC was directly correlated with the immobility time observed across animals (P<0.05 using linear regression). This relationship increased across testing sessions (P<0.05 using analysis of covariance).

The strength of IL-Thal CFPC exhibited by individual animals was directly related to their immobility time (P<0.05 for both comparisons using linear regression; illustrated at the bottom of FIG. 10A), and this relationship became stronger during the second session (F1, 24=4.51; P=0.044 using analysis of covariance). Thus, the behavioral relevance of this circuit increased with repeated testing. Directionality analysis showed that 3-7 Hz LFP activity in the IL temporally preceded oscillatory activity in Thal, providing evidence that IL-Thal CFPC reflected directional activity in the IL-Thal pathway (illustrated at the top of FIG. 10A). Together, these results confirmed that adaptations in IL-Thal circuitry corresponded with the cellular and behavioral adaptations we observed during repeated behavioral challenge.

LFP power reflects global activity patterns within a given brain region, while LFP coherence quantifies the extent to which two distinct brain regions oscillate together within a frequency across time. As such LFP coherence has generally been described as a measure of brain circuit activation. Cortical and thalamic LFP power and IL-Thal coherence (within frequency), quantified across the full group of WT animals, failed to identify differences across the testing sessions for any of the measures (F11, 286=0.68, 0.63, and 0.08; P=0.58, 0.53, 0.94, for comparison of cortical power, thalamic power, and IL-Thal coherence, respectively using repeated measures [RM] ANOVA). FIG. 10B shows temporal offsets at which IL and Thal oscillations were optimally phase synchronized at each frequency (The top of FIG. 10B shows 95% confidence interval for both testing days; N=14,). IL and Thal power, and IL-Thal coherence across testing sessions, are shown at the bottom of FIG. 10B (P>0.05 for all three measures across testing sessions using RMANOVA). Thus, the adaptations in IL-Thal function that resulted from the TST exposure were specific to the cross-spectral interactions between these brain areas.

D. IL-Thal Circuit Adaptions Are Compensatory, Not Primary

Experiments were performed to determine whether the cross-spectral neurophysiological changes observed in IL-Thal circuitry across testing sessions reflected primary or compensatory circuit adaptations. If these changes were primary (that is, if these changes included central adaptations responsible for the susceptible state that occurred in response to a behavior challenge), recapitulating this state could worsen the performance of challenge-naïve animals during behavioral challenge. Conversely, if these circuit changes were compensatory (that is, if these changes included circuit adaptations occurring in an attempt to return neural systems to ‘homeostasis’ after behavioral challenge), recapitulating them could improve the behavior of naive animals during behavioral challenge.

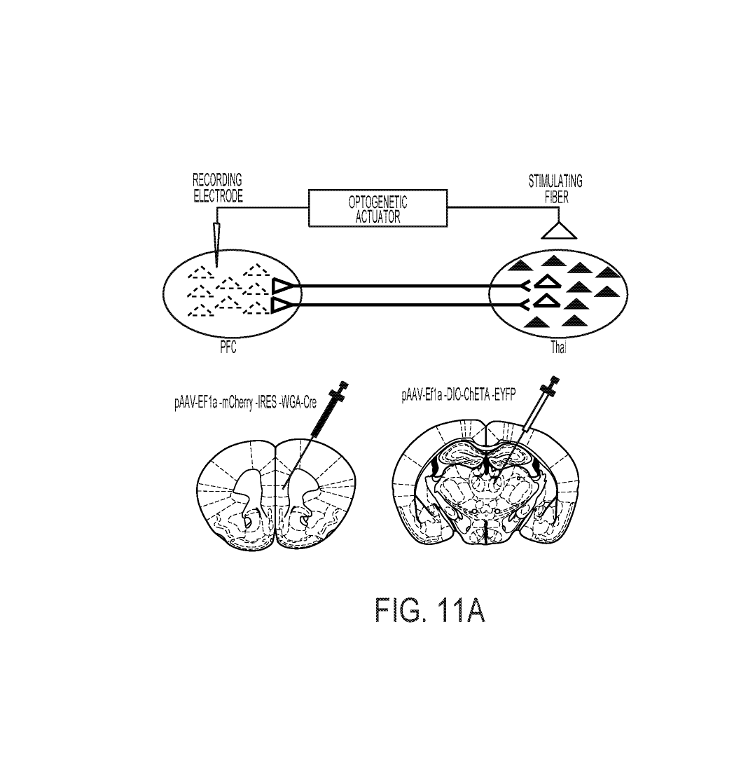

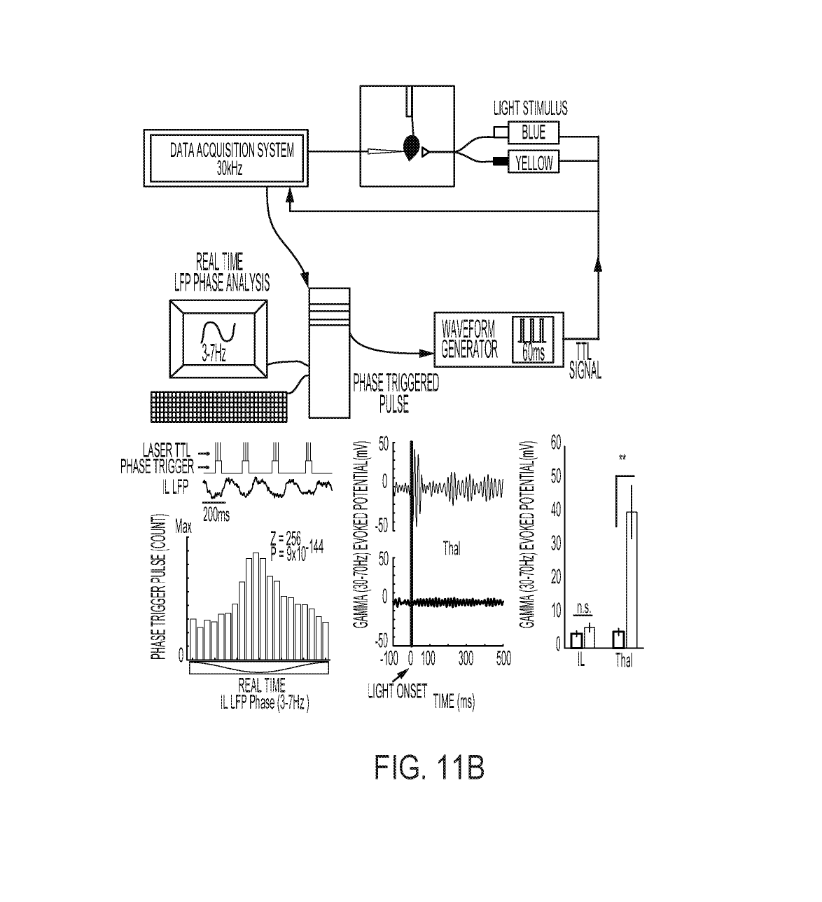

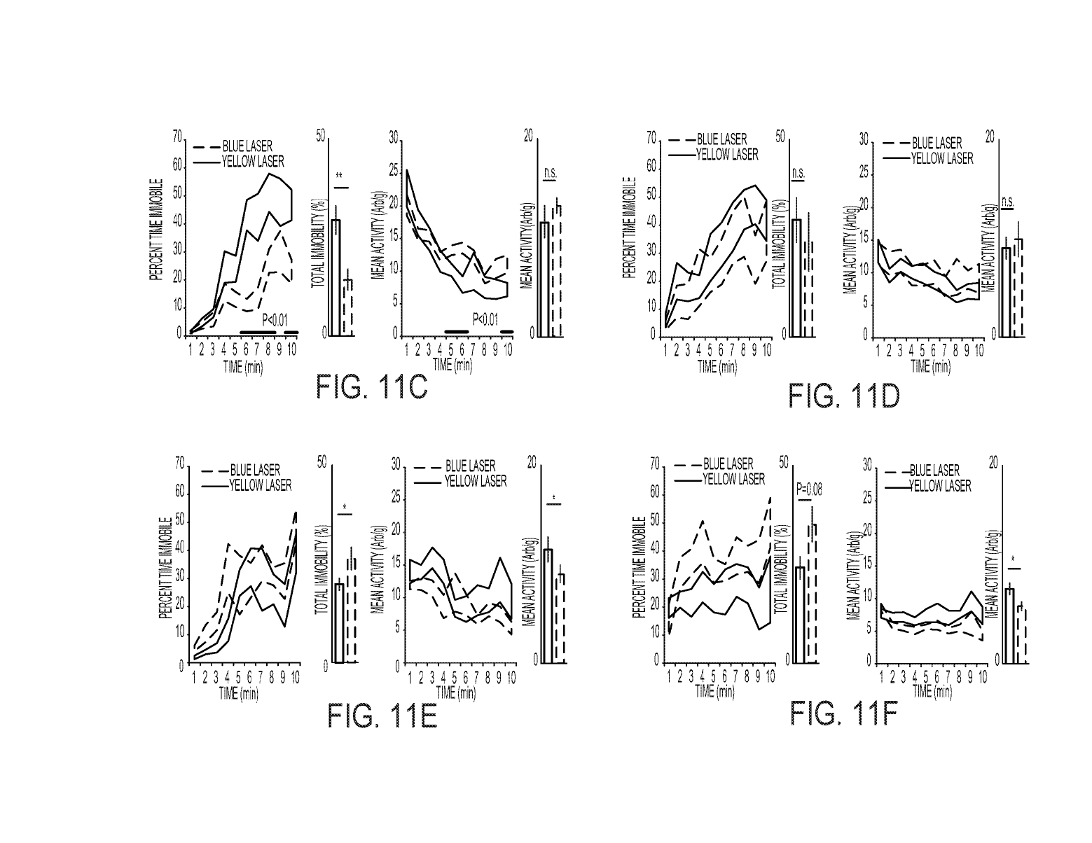

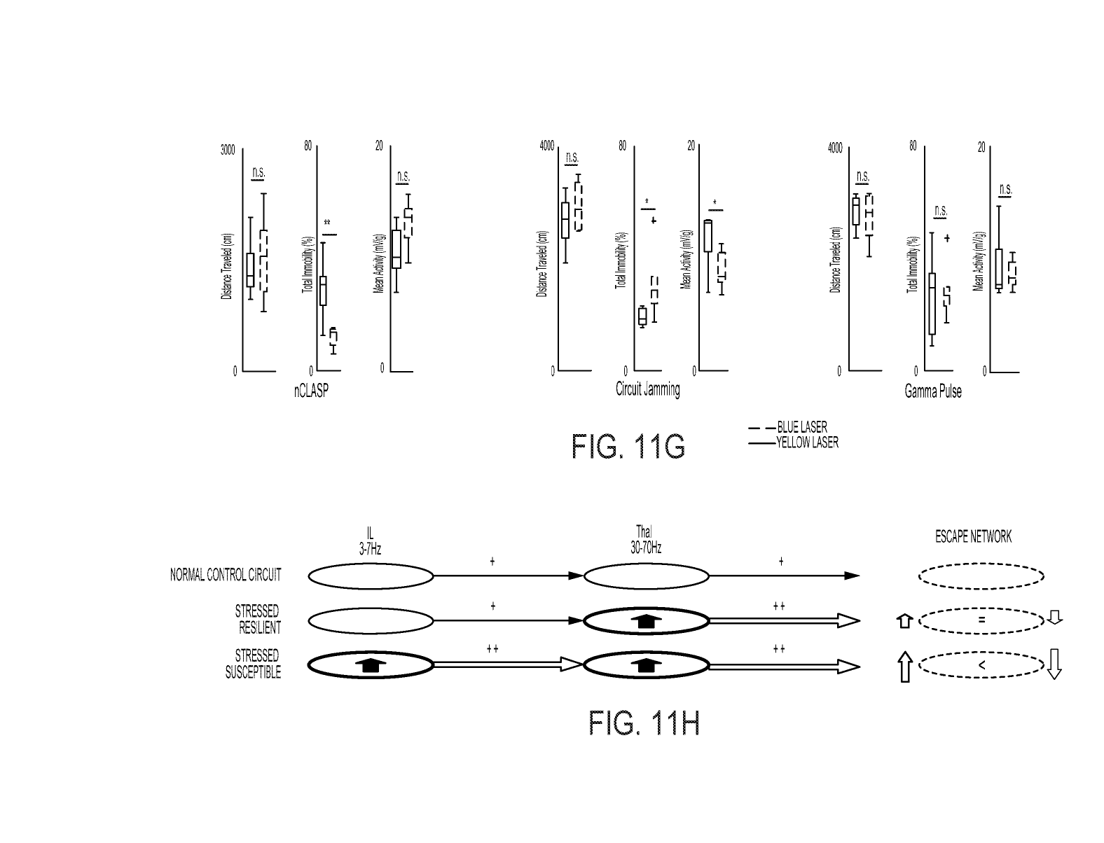

A trans-synaptic wheat-germ agglutinin-tagged Cre recombinanse (WGA-Cre) was injected into the PFC (prefrontal cortex; IL and PrL) of mice naive to behavioral challenge. Infection with WGA-Cre results in Cre expression in all neurons in the brain that form synaptic connections with PFC (both efferent and afferent synaptic connections). Thal was then infected with a floxed ultra-fast Channel Rhodopsin-2 variant (ChETA 22). These interventions are illustrated at the bottom of FIG. 11A. This strategy resulted in ChETA expression in cortical neurons that projected to thalamus and thalamic neurons that received input from PFC. EYFP expression was present in layer V/IV PFC neurons and their apical dendrites, PFC axon terminals in Thal, and the soma of Thal neurons. A neural Closed Loop Actuator for Synchronizing Phase (nCLASP) was developed to stimulate this subset of PFC axonal terminals and Thal neurons with gamma bursts timed to on-going oscillatory activity in cortex. A diagram of this stimulating system, in combination with the cells of Thal and PFC, is shown at the top of FIG. 11A.

FIG. 119 illustrates the configuration and operation of the nCLASP system to detect LFP signals from IL and to provide excitatory optical stimulation to Thal neurons based on the timing (e.g., phase) of the detected LFP signals. The nCLASP system was used to deliver light pulses to Thal at the trough of IL 3-7 Hz oscillations (shown at the bottom of FIG. 11B). Stimulation with blue, but not yellow, light evoked Thal gamma activity (shown at the bottom right of FIGS. 11B, P=0.24 and 0.0003, for comparisons of IL and Thal evoked potential amplitude, respectively, using paired unpaired t-test).

The nCLASP system facilitated driving bursts of Thal gamma activity that were phase-coupled to 3-7 Hz oscillations in cortex, paralleling the physiological parameters of the IL-Thal circuit detected during the TST. The nCLASP system was calibrated to deliver gamma bursts (3 successive 5 ms light pulses with an inter-pulse interval of 15 ms) initiated immediately before or during the rising phase of IL 3-7 Hz oscillatory cycles, as illustrated in FIG. 11B. The timing of a particular provided gamma burst was determined based on the timing of a rising phase of a preceding IL 3-7 Hz oscillatory cycle, delayed by an amount determined based on the median inter-cycle timing of a number of previous IL 3-7 Hz oscillatory cycles (e.g., a median of the inter-cycle timing of five previous inflection points in the IL 3-7 Hz oscillatory cycle). Experimental animals were stimulated with blue light to active ChETA, and control animals were stimulated with yellow light which does not activate the opsin (this is illustrated in FIG. 11B). Behavioral responses were monitored while these animals were subjected to the TST.

Induction of the IL-Thal pathway using the nCLASP system rendered the mice more tolerant to effects of behavior challenge. Specifically, mice stimulated with blue light exhibited less immobility and higher activity profiles compared to the animals stimulated with yellow light (F9, 117=3.89, P=0.008 for light effect on immobility time; F9, 117=2.73, P=0.009 for light effect on mean activity; comparisons of blue and yellow light groups were made using repeated measures ANOVA, P<0.01; P<0.05 for comparison of full test immobility time using Student's t-test. These results are illustrated in FIG. 11C, which shows the effects of nCLASP stimulation on TST behavior during initial testing sessions (bars at the bottom of each plot of FIG. 11C indicate time points where P<0.05 for post-hoc testing using paired t-test). These results are also illustrated in the “nCLASP” portion of FIG. 11G.

Experimental results demonstrated that the IL-Thal adaptations in response to prior behavioral challenge (described above) were compensatory because induction of the circuit increased escape behaviors during the behavioral challenge. No differences in gross locomotion were observed when animals were stimulated in the open field (Distance traveled: 1391±140 and 1545±232 for animals stimulated with blue and yellow light, respectively; P=0.57 using two-tailed Student's t-test) showing that stimulation of the IL-Thal pathway induced a behavioral effect that was specific to the TST paradigm. This was consistent with findings that IL neurons preferentially signal behavior in the TST compared to the open field. This is illustrated in FIG. 11D, which shows the effects of nCLASP stimulation on TST behavior during subsequent testing sessions.

Furthermore, IL-Thal stimulation had no impact on the immobility time or mean activity observed during the first four minutes of the TST (illustrated in FIG. 11C and FIG. 11G), demonstrating that this stimulation approach did not simply induce hyperactivity. Rather, stimulation of the IL-Thal pathway using the nCLASP system diminished the behavioral adaptation that occurred across the 10 minute TST session. Stimulating this circuit using the nCLASP system had no impact on behavioral responses during the TST on the subsequent day (F9, 117=0.68, P=0.68 for light effect on immobility time; F9, 117=0.92, P=0.50 for light effect on mean activity; comparisons of blue and yellow light groups were made using repeated measures ANOVA; P>0.05 for comparison of full testing immobility and mean activity using Student's t-test; this is illustrated in FIG. 11D).

Mice were also stimulated using an open loop fixed-frequency pattern. The stimulation protocol was specified to deliver the same number of light pulses as the nCLASP system (14.05 Hz, 5 ms pulse width), but in a manner not timed to oscillatory activity in IL. IL-Thal stimulation with blue light tended to diminish TST escape behavior on the first day of testing (P=0.069 and 0.14 for full test immobility time and activity, respectively, using two-tailed Student's t-test), and TST activity profiles were significantly lower in animals stimulated with blue light during the second testing session (P=0.08 and 0.027 for full test immobility and activity, respectively, using a two-tailed Student's t-test). FIGS. 11E and 11F illustrate the effects of open loop fixed-frequency stimulation on TST-behavior during initial and subsequent testing sessions, respectively. The “Circuit Jamming” portion of FIG. 11G also illustrates the effects of open loop fixed-frequency stimulation on TST-behavior during initial testing sessions.

Notably, the stimulation frequency used for our open-loop protocol was more than twice as fast as the frequency of IL to Thal input (2-7 Hz), inducing a circuit state where thalamic responses were dissociated from ongoing activity in IL (that is, the stimulation may have acted to jam the IL-Thal circuit). Thus, IL-Thal circuit stimulation using the nCLASP system induced escape behaviors, while IL-Thal jamming using open loop stimulation suppressed escape behaviors.

Mice were additionally stimulated using an open loop pattern of gamma pulses. This stimulation protocol was specified to gamma pulses at a frequency similar to the frequency of gamma pulses provided by the nCLASP system (4.68 Hz), but in a manner not timed to oscillatory activity in IL. The gamma pulses for this stimulation protocol were similar to those provided by the nCLASP system, with each gamma pulse consisting of three successive 5 ms light pulses with an inter-pulse interval of 15 ms. This pattern of IL-Thal stimulation tended to have no effect on TST behavior (tstat₁₀=0.13 and P=0.899 for full test immobility time and tstat₁₀=0.45 and P=0.659 for mean activity using two-tailed Student's t-test) or open field activity (t₉=−0.57 and P=0.582 using Student's t-test). The “Gamma Pulse” portion of FIG. 11G illustrates the effects of open loop gamma pulse stimulation on behavior.

Together these findings indicate that challenging experiences induce the IL-Thal circuit. Exogenous induction of this circuit in naive mice drove escape behaviors, while exogenous IL-Thal stimulation after the endogenous induction of this circuit (e.g., on a second day of application of the TST) has no behavioral effect. On the other hand, jamming this circuit in naïve mice using open loop stimulation had limited behavioral effects, while jamming the IL-Thal circuit after its endogenous induction suppressed escape behaviors. Taken with the cellular data obtained in the WT-resilient and WT-susceptible mice, these findings indicate that the IL-Thal pathway may operate as a compensatory regulatory circuit that activates escape behaviors during challenging experiences.

A proposed model of this system is illustrated in FIG. 11H. FIG. 11H shows a model of compensatory IL-Thal circuit adaptation occurring across sessions of the TST. In this model, the IL-Thal circuit activates a TST-escape network. In resilient animals, stress partially diminishes the output of the escape network. These animals compensate for this diminished circuit output by inducing the IL-Thal pathway. This compensation restores normal behavior. In the susceptible animals, stress profoundly suppresses the output of the TST-escape network. The compensatory increase in the IL-Thal pathway is insufficient to restore normal behavior.

This compensatory pathway is induced after challenging experiences. In normal animals that exhibit resilience to behavioral challenge, the thalamic upregulation after the initial exposure is sufficient to overcome the downstream changes and restore homeostasis in the escape network. In animals susceptible to behavioral challenge, the thalamic upregulation is insufficient to restore homeostasis in the escape network, resulting in a further upstream upregulation of infralimbic activity.

E. Behavioral Challenge Induces Network Adaptations that Alter Reward and Stress Pathways

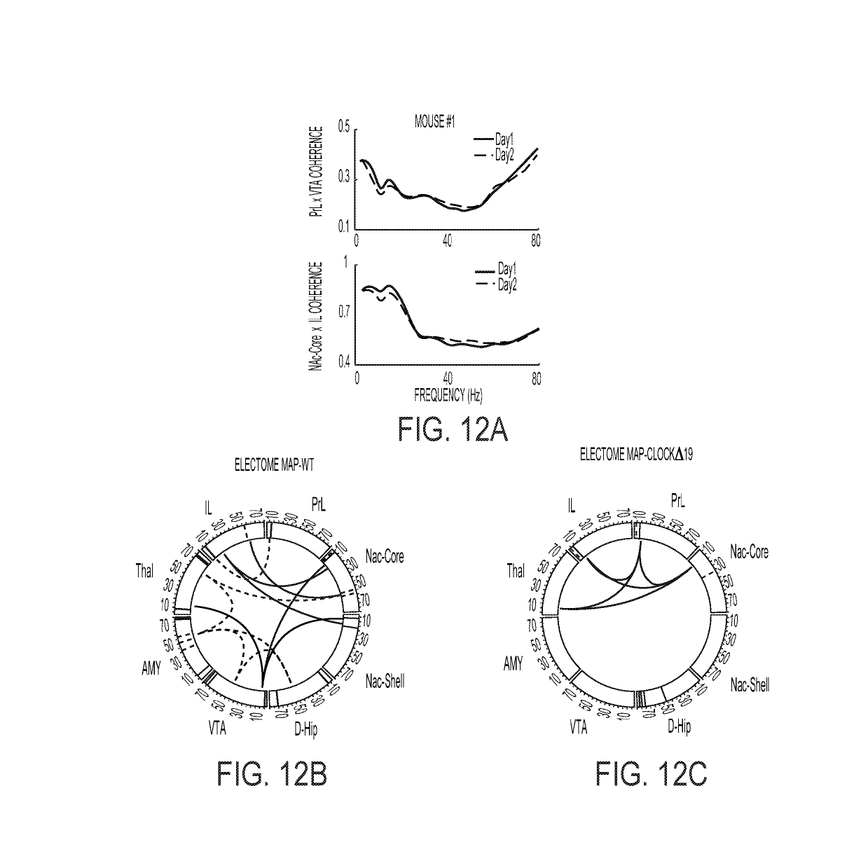

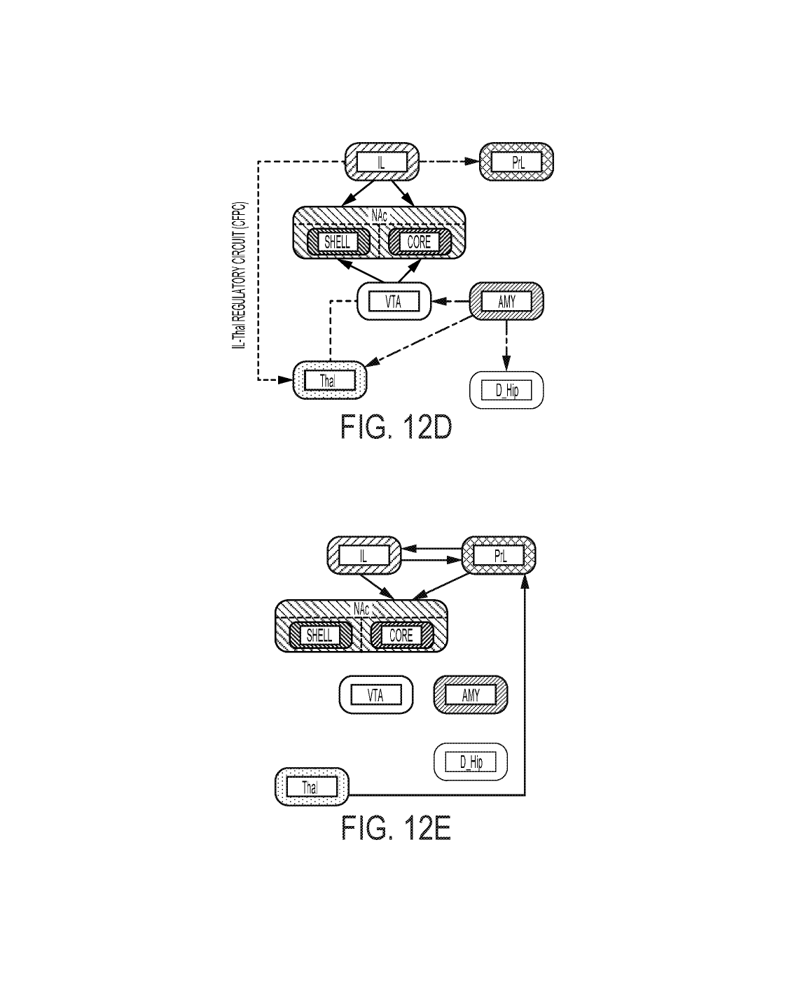

As the central prediction of this regulatory circuit adaptation model is that animals that experience the largest increase in immobility between testing sessions would also exhibit the largest decrease in the function of their TST-escape network, experiments were performed to identify any network adaptations that may parallel the immobility increases that occurred with repeat TST testing. For this analysis, a mesoscale circuit-mapping approach was employed that was based on individual differences in behavior. This approach used LFP signals obtained from a variety of brain regions during each recording session, and the analysis was performed on data recorded in the original group of 14 WT animals implanted concurrently in eight brain regions. Implementing this analysis included: 1) performing an expanded LFP analysis that included a frequency band spanning 3-80 Hz (in 1 Hz bins); 2) calculating the average LFP power within each TST session for each recorded brain region (of 8 total possible brain regions); 3) calculating the average coherence between each pair of recorded brain regions (28 brain region pairs); and 4) calculating a difference measure for each determined power and coherence measure between the two TST testing sessions (that is, between the initial TST session and the subsequent TST session). FIG. 12A illustrates determined mean LFP coherence values calculated across frequency for two example pairs of brain regions. This approach yielded 2808 calculated difference measures for each animal (determined from 8 brain regions, 28 brain region pairs, and 78 frequency bins).

A multivariate regression was performed, using the Elastic Net algorithm, to determine which patterns of these 2808 difference measures best accounted for the change in immobility each animal showed across the two TST sessions. The Elastic Net algorithm is a regularized approach based in supervised machine learning. It is particularly suitable for analyses in which the predictor variables are highly correlated with each other (such as in the case of brain oscillations), and yields a model in which each of the predictors that returns a non-zero regression coefficient contributes to the multivariate model solution. Using, this approach, an Electrical Functional Connectome (hereafter referred to as Electome) was determined to describe patterns in neural activity adaptations that were linked to stress induced behavioral changes observed in the experiments.

Several of the coherence measures retained in the Electome mapped directly onto structural pathways widely shown to regulate motivated behavior or responses to challenging experiences. Specifically, it was found that repeated TST exposure decreased the coherence in canonical reward circuits (e.g., VTA to NAc) and increased coherence in circuits implicated in mediating stress and anxiety responses (e.g., AMY to D-Hip). Thus, multiple circuit elements that drive motivated behaviors were altered in the animals that showed the largest increase in immobility time across testing sessions. It was also found that the animals exhibited a decrease in theta (7-12 Hz) power in all of the recorded brain regions and an increase in low-gamma (30-70 Hz) power in AMY, NAc-Core, and IL. Taken together, these results indicate that the power changes that occurred with repeated TST testing were global, while the changes in coherence were limited to specific neural circuits.

The Electome determined for the wild-type mice is illustrated in FIG. 12B. FIG. 12B illustrates the difference measures retained in the Elastic Net solution for wild-type mice. Ribbons that cross the circle signify coherence measures that were retained in the solution, and bands indicated at the rim of the circle signify power measures that were retained. Difference measures with negative coefficients (that is, that were negatively correlated with change in immobility) are shown in solid lines, and predictors with positive coefficients are shown in dashed lines. Labels around the circle plot indicate the brain area and frequency (3-80 Hz) for each difference measure.

Directionality analysis was performed on the coherence predictors retained in the Electome and the results were integrated into a single network model. This model is illustrated in FIG. 12D. The IL-Thal regulatory circuit and the Thal-VTA integrator circuit are shown in FIG. 12D as well. This analysis revealed that the network adaptation that followed repeat TST exposure was organized by two nodes: one in VTA and another in IL. The output of both of these nodes converged in NAc, and it was diminished in the mice that showed the largest increases in immobility. It was also found that the coherence between Thal and VTA decreased in these mice. Though this coherence measure did not exhibit directionality, it mapped on to the same oscillatory frequency that was suppressed in the VTA to NAc circuit. The Thal-VTA pathway was situated between the IL-Thal regulatory circuit and the VTA to NAc circuit, ultimately linking the two TST-escape network nodes. Since there are no direct projections from Thal to VTA, this pathway likely involves polysynaptic relays through a third unknown brain circuit.

Critically, several circuit adaptations involving IL, but not VTA, were also observed in the Electome for Clock-Δ19 mice, showing that the neural adaptations impacting the IL TST-node were not central to the behavioral change that occurred in WT mice across testing sessions. The Electome determined for the Clock-Δ19 mice is illustrated in FIG. 12C, which illustrates the difference measures retained in the Elastic Net solution for the Clock-Δ19 mice. The model determined from a directionality analysis performed on the coherence predictors retained in the Clock-Δ19 Electome is illustrated in FIG. 12E.

F. The VTA TST-Node is Modulated by 5-HT Stimulation

The change in TST immobility displayed by individual animals appeared to be due to a suppression of their escape network. Thus, the measures retained in the Electome mirrored this escape network. As selective stimulation of 5-HT neurons in dorsal raphe increases escape behavior in mice exposed to the forced swim test, experiments were performed to test whether direct stimulation of 5-HT neurons in dorsal raphe stimulated the discovered escape network described above. Specifically, experiments were performed to determine if 5-HT stimulation induced a network state in the VTA-node that opposed the adaptations observed in the Electome model after repeated TST exposure.

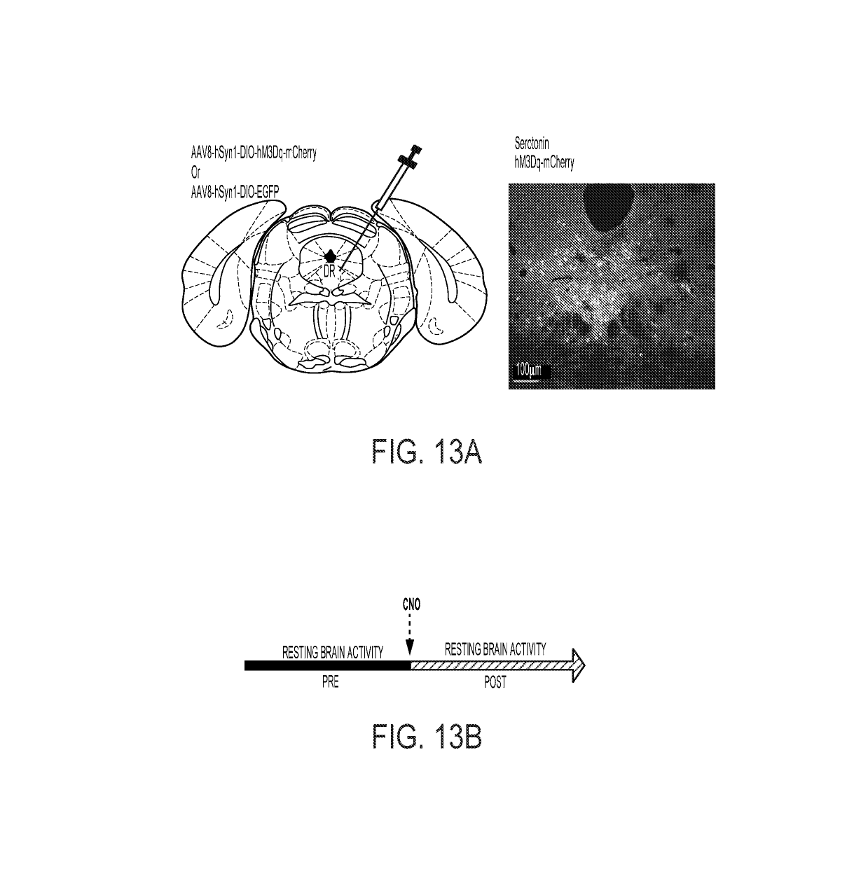

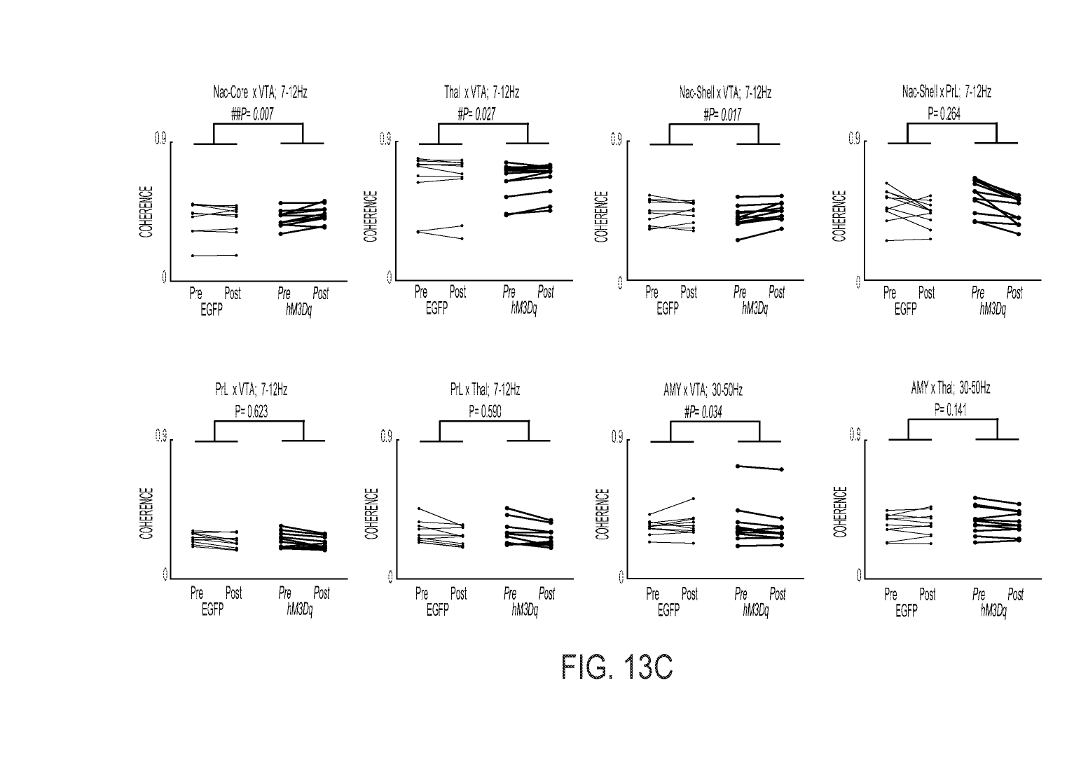

Slc6a4-Cre mice were infected with a floxed hM3Dq-DREADD (N=11 mice) or a floxed control EGFP virus in dorsal raphe (N=10 mice). This approach resulted in the expression of the hM3Dq-DREADD in dorsal raphe 5-HT neurons. This intervention and its results are illustrated in FIG. 1A, with the left part of FIG. 13A showing the protocol for DR 5-HT viral infection strategy and the right part of FIG. 13A showing a histological image illustrating the location of 5-HT and/or hM3Dq-mCherry expression.

Following surgical recovery, mice were implanted with recording electrodes to target the VTA-node (VTA, Thal, NAc-Core, NAc-Shell, and AMY) and to target PrL as a control area, Neural recordings were obtained while animals were in their home cage prior to and 45 minutes after treatment with CNO (2 mg/kg), which activates the DREADD (a schematic diagram of the timing of these experiments is illustrated in FIG. 13B). Resting network responses in the DREADD and EGFP expressing mice were then compared. FIG. 13C illustrates these calculated resting coherence measures within the VTA-node and across PrL-dependent circuits prior to and following treatment with CNO (2 mg/kg). P<0.05 for the effect of the virus by drug treatment using mixed model ANOVA.

Activation of 5-HT neurons induced a network state in the VTA-node that opposed the adaptive changes that accompanied repeat TST exposure. Specifically, 5-HT activation increased VTA×NAc-Core, VTA×NAc-Shell, and VTA×Thal theta coherence, and decreased AMY×VTA gamma coherence (P=3.9×10−7 [0.025̂4] being the calculated likelihood that all four of these changes would be observed in response to 5-HT activation by chance). No changes were observed in AMY×Thal gamma coherence. Furthermore, no changes were observed in PrL×VTA or PrL×Thal theta coherence, showing that the network changes induced by DR 5-HT neuron stimulation selectively impacted activity in the VTA-node. Thus, these findings confirmed that activity in the node shared a mesoscale-level mechanistic overlap with the convergent neural pathway targeted by antidepressants. Furthermore, these findings showed that the neural adaptations observed in the VTA-node after repeat TST exposure play a central role in mediating the increases in immobility exhibited by mice.

Stress contributes to the onset and exacerbation of nearly all mental disorders. The TST is widely used as a preclinical model of major depressive disorder due to the assay's sensitivity to acute treatment with clinically relevant antidepressants. While immobility during the test may not fully recapitulate the range of symptoms observed across this complex disorder, it is striking that the distinct VIA-node adaptations that occur in response to repeated TST exposure share a clear mechanistic overlap with the convergent molecular pathway targeted by antidepressants. The sensitivity of the TST escape behavior to acute antidepressant treatment is likely due to this circuit overlap.

Prior studies have shown that the direct optogenetic manipulation of the activity of VTA dopamine neurons is sufficient to bi-directionally regulate escape behavior during challenging experiences. These optogenetic findings support the hypothesis that maladaptive behavioral responses after challenging experiences may result from a downregulation of cellular activity in the VTA. In contrast, the cellular and. Electome mapping findings presented here suggest that local decreases in VTA activity are insufficient to explain the behavioral adaptation that occurs in response to repeated TST testing.

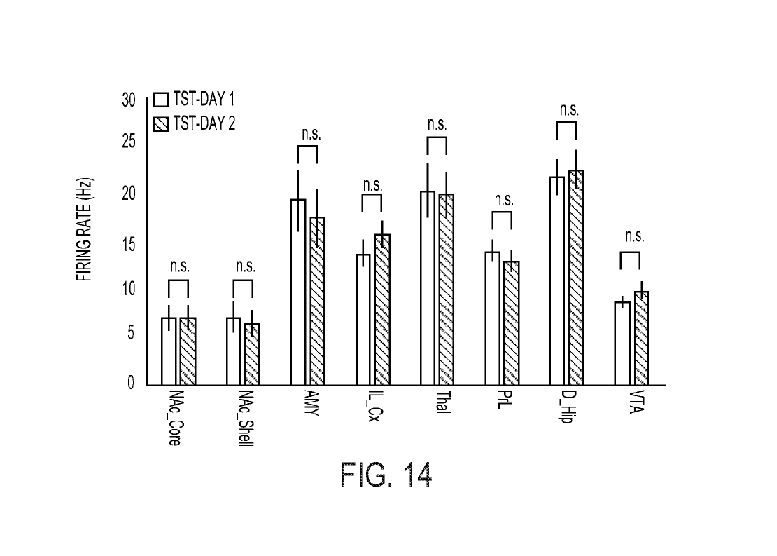

First, a suppression of VTA cellular responses across testing sessions was not observed. This is illustrated, for example, in FIGS. 9A, 9B, and FIG. 14. FIG. 14 shows that no changes in mean unit firing rates were observed across the two TST sessions (P>0.25 for all comparisons using rank-sum test). Additionally, while decreased LFP power in the VTA accompanied TST adaptation, similar local oscillatory power changes were observed in nearly all of the monitored brain regions. Thus, the supraphysiological optogenetic stimulation/inhibition of VTA dopamine neurons may directly alter activity within the mesoscale networks that regulate responses to behavioral challenges, but the physiological mechanisms that underlie the emergence of pathological behavioral changes are unlikely to result from global suppression of activity in the VTA.

These findings suggest that the network level adaptations found in the VTA-node may reflect the true neurophysiological substrate underlying maladaptive behavioral changes. These mesoscale adaptations in the VTA-node were opposed by 5-HT stimulation, and were not observed in the Clock-Δ19 mice. These mesoscale adaptations are unlikely to directly map onto cellular changes within a single brain area, as both increases and decreases in coherence between VTA and other subcortical brain regions accompanied the behavioral changes that followed repeat TST exposure.

The Electome mapping findings also showed that exposure to challenging experiences (e.g., to the TST) results in the disruption of a Thal-VTA to NAc circuit. Taken together with the closed-loop optogenetic stimulation experiments (which showed that stimulation of the IL-Thal pathway is sufficient to induce escape behaviors prior to but not following repeat stress), these findings suggest that this Thal-VIA functional pathway may be a central network component that is dysregulated by stress ultimately resulting in decreased Thal to VTA (indirect) signaling and driving compensatory changes in Thal and IL cellular function. Indeed, functional changes involving a thalamic network node have been described in subjects with stress-related behavioral dysfunction such as major depression.

Experimental observations indicate that exposure to behavioral challenge results in the compensatory functional expansion of IL. This functional expansion of the neocortex reflects a network state in which increased neuronal resources are required to achieve similar behavioral responses during challenging experiences (illustrated, e.g., in FIGS. 9A and 9 B). The functional expansion of IL observed with repeat TST exposure overlapped with the functional anatomical and network-level mechanisms implicated in mood regulation and depression in humans (IL is most commonly suggested as the rodent anatomical equivalent of subgenual cingulate cortex in humans based on anatomical connections). Direct electrical stimulation of subgenual cingulate cortex has been shown to ameliorate depressive symptoms in subsets of patients otherwise resistant to treatment, and direct electrical stimulation of IL reduces immobility time in rats subjected to a similar stress-related behavioral assay (e.g., the forced swim test). Since recent findings have shown that direct dysregulation of IL activity is sufficient to disrupt VTA-NAc circuit function and induce anhedonia, these findings suggest that the compensatory changes in IL that result from the stress-induced dysregulation of VTA-dependent circuitry may be sufficient in and of themselves to induce dysfunction across distinct behavioral domains. While the VTA-dependent circuitry may be the target of acute antidepressant treatment, chronic treatment may be necessary to reverse the compensatory changes that occur in feed-forward IL-dependent circuitry and the distinct behavioral changes that result from this adaptation.

Overall, these findings suggest that neural adaptations that alter mesoscale processing across (and not simply cellular activity within) brain regions may serve as common neural circuit mechanisms whereby stress exposure promotes behavioral pathology. These network adaptations then induce a compensatory upregulation of localized cellular responses in intralimbic cortex in order to restore circuit homeostasis. These localized signals likely underlie the imaging findings commonly described in human studies, and may ultimately contribute mechanistically to the manifestation of secondary behavioral disruptions such as anhedonia.

The nCLASP stimulation system induced escape behaviors in naïve mice during the TST, while stimulation using a standard open-loop protocol that delivered an equivalent number of light pulses tended to have the opposing effect (that is, tended to have a ‘circuit jamming’ effect). This finding showed that the neural state timing at which stimulation is delivered plays a critical role in determining the impact of cellular activation on behavior. This principle has particularly profound implications for developing DBS based therapies for stress induced disorders, and for interpreting the link between specific cell types and behavior given the vast body of literature in which various open loop optogenetic stimulation protocols have failed to elicit behavioral effects.

G. Supplementary Figures

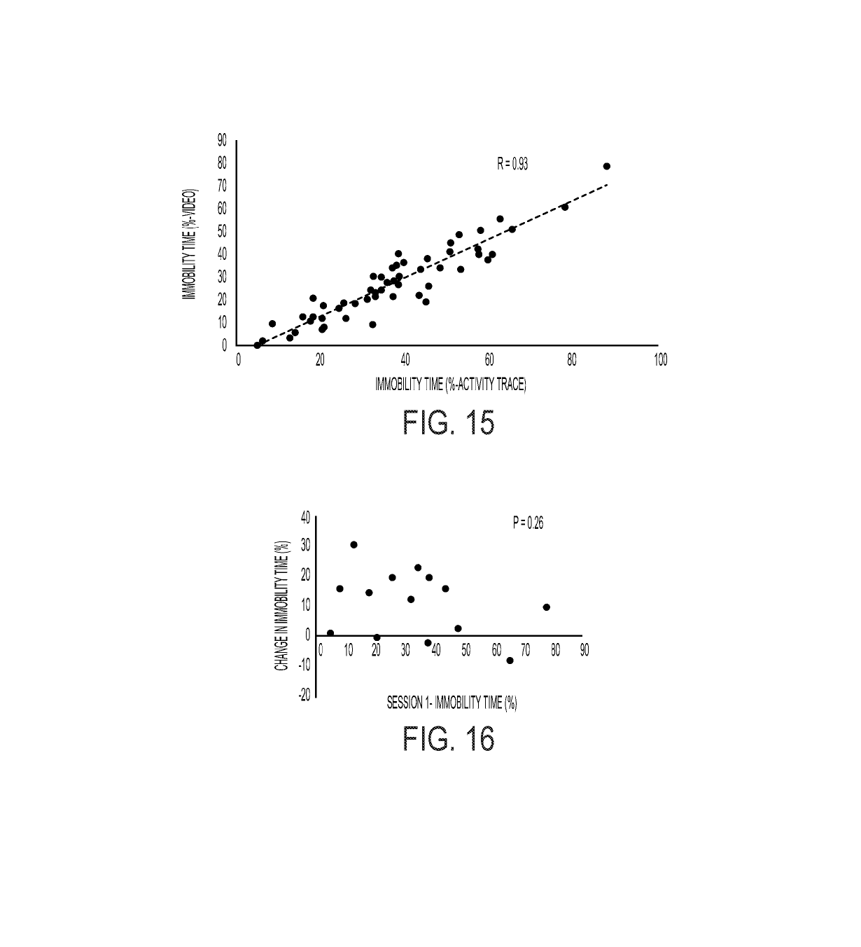

FIG. 15 illustrates the relationship between automated scoring of tail suspension test immobility and classic video scoring. Video was scored for immobility in 1 second intervals (P<0.0001, R=0.93 using linear regression). Note that automated scoring based on the electrical activity trace was more sensitive to changes in movement.

FIG. 16 illustrates that behavioral changes between tail suspension test sessions was independent of immobility during initial testing session in wild-type mice (P=0.26 using linear regression)

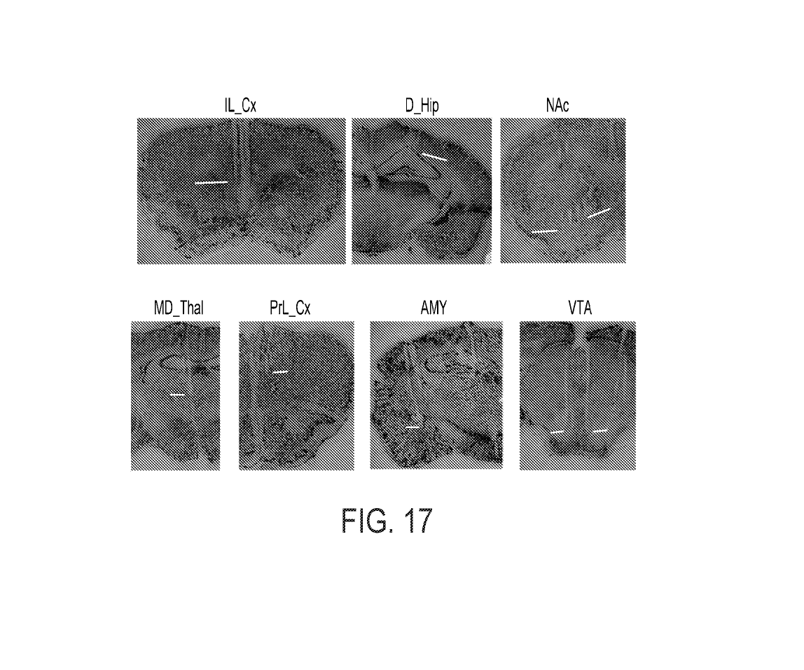

FIG. 17 illustrates example lesion tracks showing electrode localization. White lines highlight microwire tips.

H. Animal Care and Use

Clock-Δ19 mice were created by N-ethyl-N-nitrosurea mutagenesis and produce a dominant-negative CLOCK protein as previously described. Mice used for TST recording experiments were bred on from heterozygous (Clock^{Δ19}/−) breeding pairs on a BALB/CJ and C57BL/6J mixed strain background. Male Clock-Δ19 (Clock^{Δ19}/Clock^{Δ19}) and WT (+/+) littermate controls were used for all electrophysiological recording experiments presented in this study. Inbred BALB/cJ male mice (strain: 000651) purchased from the Jackson Labs were used for optogenetic stimulation and nCLASP experiments. Twenty-one twelve to sixteen week old male Slc6a4-Cre mice (MMRRC: Stock number-017260UCD) were used for DREADD experiments. Mice were housed three to five per cage on a 12-hour light/dark cycle, and maintained in a humidity- and temperature-controlled room with water and food available ad lihit; an, Behavioral and electrophysiological experiments were conducted during the light cycle (Zeitgeber time: 4-12). All studies were conducted with approved protocols from the Duke University and University of North Carolina Institutional Animal Care and Use Committees and were in accordance with the NIH guidelines for the Care and Use of Laboratory Animals.

I. Electrode Implantation Surgery

At an age of 4-9 months, WT and Clock-Δ19 mice (N=27) were anesthetized with isoflurane (1.5%), placed in a stereotaxic device, and metal ground screws were secured to the cranium. A total of 64 tungsten microwires were arranged in array bundles and implanted in Nucleus Accumbens (NAc-Core and NAc-Shell), Prelimbic cortex (PrL), Infralimbic Cortex (IL), Medial dorsal thalamus (MD-Thal), Ventral Tegmental Area (VTA), Amygdala (AMY), and dorsal hippocampus (D-Hip) based on stereotaxic coordinates measured from bregma (NAc-Core: 1.2 mm antero-posterior (AP), 1.25 mm mediolateral (ML), −3.9 mm dorsoventral (DV); NAc-Shell: 1.0 min AP, 1.70 mm ML, −4.25 min DV; MD-Thal: −1.5 mm AP, 0.35 mm ML, −2.88 mm DV; AMY: −1.58 mm AP, 2.85 mm ML, −3.90 mm DV; PrL: 1.8 mm AP, 0.125 mm ML, −1.75 mm DV; IL: 1.8 mm AP, 0.125 mm ML, −2.25 mm DV; D-Hip: −1.70 mm AP, −1.25 mm ML, −1.3 mm DV; and VTA: −3.4 mm AP, 0.25 mm ML, −4.25 mm DV; all DV coordinates measured from the dura). Several microwires were also implanted to target Substantia Nigra (−3.08 mm AP, −1.25 mm, ML, −4.25 mm DV) and Lateral Habenula (−1.5 mm AP, −0.35 mm ML, 1.8 to −2.38 mm DV), though these brain areas were not analyzed as part of this study. Implanted electrodes were anchored to ground screws above anterior cranium and cerebellum using dental acrylic. Experiments were initiated following a 4-6 week recovery. All recording sites were confirmed histologically at the conclusion of experiments.

J. Behavioral Testing

Headstages were connected without anesthesia, and animals were habituated to the recording room for 90 minutes prior to testing. All behavioral testing was conducted under low illumination conditions (1-2 lux). Mice were initially placed in a 17.5 in×17.5 in×11.75 in (L×W×H) chamber for five minutes of open field testing. The location of the animals was acquired in real time using the NeuroMotive system (Blackrock Microsystems, Inc., Salt Lake City, Utah). Mice were then transferred to a tail suspension test (TST) apparatus (Med Associates, St. Albans, Vt. MED-TSS-MS) that was modified to allow for continuous acquisition of animal motion. Mice were suspended 1 cm from the tip of their tail for ten minutes. The activity trace was digitized at 2000 Hz and stored in real time with recorded neurophysiological recording data. Open field and TST neurophysiological data was acquired during a single testing session, and the behavior testing session was repeated the next day. The quality of video tracking was confirmed offline using the NeuroMotive system.

K. Neurophysiological Data Acquisition

Neurophysiological recordings were performed during the open field and TST. Neuronal activity was sampled at 30 kHz, highpass filtered at 500 Hz, sorted online, and stored using the CerePlex Direct acquisition system (Blackrock Microsystems Inc., Utah). Neuronal data were referenced online against a wire within the same brain area that did not exhibit a signal to noise ratio greater than three to one. At the end of the recording, cells were sorted again using an offline sorting algorithm (Plexon Inc., Tex.) to confirm the quality of the recorded cells. Local field potentials (LFPs) were handpass filtered at 0.5-250 Hz and stored at 1000 Hz. All neurophysiological recordings were referenced to a ground wire connected to both ground screws. Video recordings were acquired in real time using the NeuroMotive system, and synchronized with neurophysiological data.

L. Generating OF and TST Behavioral Activity Traces

Video was acquired at 50 frames per second. Spatial coordinates were extracted for each video frame, and the instantaneous velocity for each frame was then calculated based on the distance animals traveled during the 180 ms window surrounding each video frame. A continuous velocity trace was then generated under the assumption that acceleration was constant between successive video frames. TST activity was smoothed over a 100 ms window with 1 ms step. All data was averaged into 1 s second bins for the LFP analysis or 50 ms bins for the unit analysis.

M. Construction of Peri-Event Time Histograms

Movement onset was characterized by 500 ms of activity below the movement threshold followed by a 250 ms interval in which the first activity measure and the 99% confidence of the remaining interval was above the movement threshold. Note that these data were not used for the main neural analysis presented herein.

N. Modeling Spike-Behavioral Activity Relationship

TST and open field test behavioral traces were centered and standardized for each recording session prior to spike analysis. The spiking data was separated into 50 ms bins. yₜ represents the value of the behavior trace at time t and yₜ represents the number of spike counts for a single unit at time t. The data is split into 30 contiguous time segments, and 15 time segments are chosen at random for each neuron to train the parameters, and the remaining 15 time segments are used as a validation set.

The model uses a linear relationship to predict the behavior trace from the spiking data. Letting dₜ represent the parameters, and εₜ represent additive Gaussian noise, the model is written as:

yₜ=Σ_{l=-L}^{L}dₗxₜ₋ₗ+εₜ

The algorithm maximizes the penalized log-likelihood (which corresponds to the maximum a posterior (MAP) solution of a Bayesian model with priors

$$d ∼ N ( 0 , \frac{{σ}^{2}}{λ} l )$$

and εₜ˜N(0, σ²I)), which corresponds to maximizing:

Σₜ₌₁^{T}(yₜ−Σ_{l=-L}^{L}dₗxₜ₋ₗ)²+λΣ_{l=-L}^{L}dₗ²

L was set to correspond to 500 ms. The hyperparameter λ is chosen individually for each spike train by using cross-validation on the training set, and performance is evaluated by calculating the reduction in fractional error (RFE) in the prediction of the hold-out behavior trace. Fractional error is normalized Mean Squared Error, and is defined as

1−(Σₜ₌₁^{T}(yₜ−Σ_{l=-L}^{L}dₗxₜ₋ₗ)²)/Σₜ₌₁^{T}yₜ²

An RFT greater than 0 corresponds to a positive predictive relationship on the previous unseen hold-out time series data. These methods are standard in the literature, and similar to the model in Paninski et al. The implementation details and extensions have been previously published. Only neurons with an RFE greater than 0.03 were used for analysis based on predictive waveforms. The effect of stress exposure on neuronal response profiles (e.g. portion of recorded neurons with RFE greater than zero) was analyzed across testing sessions using a G-test with a Williams' correction.

O. LFP Oscillatory Power and Cross-Area Coherence

Signals recorded from all of the implanted microwires were used for analysis. Using Matlab (The MathWorks, Inc., Natick, Mass.), a sliding Fourier transform with Hamming window was applied to the LFP signal using a 1 second window and a 1 second step. Frequencies were analyzed with a resolution of 1 Hz. The LFP oscillatory power values used for analysis were then assigned as the mean power observed across all of the LFP channels for a given brain area. LFP cross-structural coherence was calculated from each cross area microwire LFP pairs using magnitude-squared coherence

$${C}_{AB} ( f ) = \frac{{\vert {Psd}_{AB} ( f ) \vert}^{2}}{{Psd}_{AA} ( f ) {Psd}_{BB} ( f )}$$

where coherence is a function of the power spectral densities (PSDs) of A and B, and their cross-spectral densities. These calculated coherence values were then averaged across all of the microwire pairs recorded from a given pair of brain areas. This final coherence value was used for analysis. One Clock-Δ19 mouse exhibited poor neurophysiological signal quality from wires implanted in AMY. This animal was excluded from the LFP data analysis. For the other animals, data were imputed for 1 s activity bins that exhibited LFP signal saturation (0.59±0.1% of the total time points recorded, with an average of 3.6 seconds per animal for the 600 s TST recording session). Finally, all data was averaged within each session.

P. Optogenetic Viral Infection Surgeries

At an age often-twelve weeks, BALB/cJ mice were anesthetized with isoflurane, placed in a stereotaxic device, and injected with AAV2-EFla-mCherry-IRES-WGA-Cre (WGA-Cre) based on stereotaxic coordinates measured from bregma at the skull (IL: 1.70 mm AP, 0.72 mm ML, −2.03 mm DV at a 10° angle). Mice were also injected with pAAV2-Efla-DIO-ChETA-EYFP (DIO-ChETA) based on stereotaxic coordinates measured from bregma (Thal: −1.58 mm AP, 0.50 mm ML, −2.88 DV at a 10° angle). A total of 0.6 μL of each virus was delivered unilaterally at each injection site over five minutes using a 5 μL Hamilton syringe. All viruses were obtained from the UNC Gene Therapy Center (Chapel Hill, N.C.; courtesy of K. Deisseroth). Thirteen of these animals were also implanted with optic fibers to target Thal based on stereotaxic coordinates measured from bregma (fiber tip placement: 1.58 AP, 0.5 mm ML, −2.38 mm DV). The remaining animals were allowed to recover at least 3 week prior to implantation of the microwire opirode construction. The microwire electrode array was designed to target IL and Thal. A Mono Fiberoptic Cannula coupled to a 2.5 mm metal ferrule (NA 0.22; 100 μm [inner diameter], 125 μm buffer [outer diameter], MFC_100/125-0.22, Doric Lenses, Quebec) was built directly into the Thal bundle. The tip of the fiber was secured 500 μm above the tip of the tungsten microwires. Optogenetic experiments were initiated in both groups 6-7 weeks after viral infection. Note that AAV2 viruses have been shown to exhibit anterograde infection in the thalamus at this time frame.

Q. Immunohistochemistry For Optogenetic Studies

Immunostaining and confocal imaging were performed. Briefly, animals were anesthetized with isoflurane and transcardially perfused with PBS followed by ice-cold 4% paraformaldehyde. The brains were quickly dissected out and post-fixed in 4% paraformaldehyde overnight at 4 C. After the post-fixation, the samples were further cryoprotected for 2 days in 30% sucrose/PBS at 4 C and embedded in the Optimum Cutting Temperature (OCT) compound (Tissue-Tek). The brains were sliced at 80 microns, then incubated in blocking buffer (PBS containing 10% Blocking One (Nacalai Tesque Inc.), and 0.3% Triton-X100) for 1 hour at room temperature with rabbit anti-GFP antibody (1:1000, ab290, Abcam) overnight at 4 C. The sections were washed and then incubated with donkey anti-rabbit secondary antibody (Alexa Fluor 488, Jackson ImmunoResearch) and counter stained with DAPI. Images were taken with Zeiss 700 laser scanning confocal microscope at ×10 objective lens.

R. hM3Dq Viral Infection Surgeries

Slc6a4-Cre mice (MMRRC: Stock number-017260UCD) were used for all DREADD experiments. Mice were anesthetized with Ketamine (100 mg/kg) and xylazine (10 mg/kg), place in a stereotax, and injected with 0.75-1 μl of AAV8-hSyn1-DIO-hM3Dq-mCherry or AAV8-hSyn1-DIO-EGFP bilaterally based on stereotaxic coordinates measured from bregma at the skull (Dorsal Raphe: −4.60 mm AP, ±1.93 mm ML, −3.46 mm DV at a 30° angle). Two weeks following surgical recovery, mice were again anesthetized with Ketamine (1.00 m/kg) and xylazine: (10 mg/kg) and implanted with microwire array recording electrodes in NAc-Core, NAc-Shell, PrL-Cx, Thal, VTA, and AMY based on stereotaxic coordinates measured from bregma (NAc-Core: 1.2 mm AP, 1.00 mm ML, −3.9 mm DV; NAc-Shell: 1.0 mm AR 0.50 mm ML, −4.20 mm DV; Thal: −1.5 min AP, 0.25 mm ML, −2.88 mm DV; AMY: −1.58 mm AP, 2.85 mm ML, −3.90 mm DV; PrL: 2.6 mm AP, 0.25 mm ML, −0.80 mm DV and VTA: −3.4 mm AP, 0.25 mm ML, −4.25 mm DV; all DV coordinates measured from dura) Neurophysiological recordings were conducted 3 weeks after surgical recovery.

S. Immunohistochemistry For 5-HT Studies

Mice were anesthetized with tribromoethanol and then perfused with 4% paraformaldehyde (PFA) in PBS. Brains were dissected and postfixed overnight at 4° C. in 4% PFA, followed by dehydration in a 30% sucrose solution in PBS for 48 h. Brain sections (40 microns) were permeabilized for 20 min with 0.3% Triton X-100 in PBS and then blocked in blocking buffer (PBS containing 0.3% Triton X-100, 2% normal goat serum, and 3% bovine serum albumin) for 1 h at room temperature. Slides were incubated with primary antibody in blocking buffer overnight at 4° C. Primary antibodies used in the studies were: anti-RFP (mouse, 1:1000, Abcam anti-5-HT (rabbit, 1:500, Immunostar), and anti-GFP (mouse, 1:500, Abcam). Slides were washed and incubated with secondary fluorescent-conjugated antibodies (1:250, Invitrogen) at room temperature (RT) for 1 h. After an additional three washes, slides were mounted and fluorescent images were collected on an Olympus Fluoview FV1000 Laser Scanning Confocal Microscope (Olympus). The images of coronal slices were analyzed using the Image-4 software (NIH).

T. Determination of LFP Cross Frequency Phase Coupling (CFPC)

LFPs were filtered using 3^{rd} order Butterworth bandpass filters designed to isolate 0.5 to 9.5 Hz IL oscillations (in 1 Hz increments for phase analysis) and 15 to 70 Hz Thal oscillations (in 5 Hz increments for amplitude analysis). The instantaneous amplitude and phase of the filtered LFPs were then determined using the Hilbert transform, and the modulation index was calculated using the MATLAB code provided by Canolty et al. Briefly, a continuous variable z(t) is defined as a function of the instantaneous theta phase and instantaneous gamma amplitude such that z(t)=A_{G}(t)*e^{iϕ}_{TH}^{(t)}, where A_{G} is the instantaneous gamma oscillatory amplitude, e^{iϕ}_{TH} is a function of the instantaneous theta oscillatory phase. A time lag is then introduced between the instantaneous amplitude and theta phase values such that zₛᵤᵣᵣ is parameterized by both time and the offset between the two variables, zₛᵤᵣᵣ=A_{H}(t+τ)*c^{iϕ}_{TH}^{(t)}. The modulus of the first moment of z(t), compared to the distribution of surrogate lengths, provides a measure of coupling strength. The normalized z-scored length, or Modulation index, is then defined as M_{NORM}=(M_{RAW}−μ)/σ, where M_{RAW} is the modulus of the first moment of z(t), μ is the mean of the surrogate lengths, and σ is their standard deviation.

After determining the optimal bands for phase-amplitude coupling, IL LFPs were filtered using 3^{rd} order Butterworth bandpass filters designed to isolate 3 to 7 Hz oscillations, and Thal LFPs were filtered to isolate 30 to 70 Hz oscillations. The instantaneous amplitude and phase of these filtered LFPs were then determined using a Hilbert transform. Since limbic cross-frequency phase coupling is strongly affected by movement, only LFP data extracted from TST intervals in which animals were immobile were used for CFPC analysis. Modulation index values were calculated for half of the microwire IL-Thal pairs, and data were averaged across these LFP pairs for each session.

U. Extraction of Real-Time LFP Phase

Online LFP activity was sampled from infralimbic cortex at 10 kHz and captured in a 500 ms sliding window. This sliding LFP window was updated with real time activity every 15 milliseconds, and a 2^{nd} order Butterworth band pass filter designed to isolated activity in the 3-7 Hz range was applied to the updated 500 ms LFP sample. A Hilbert transform was applied to the last 100 ms of this sliding LFP window, and the phase at the 500^{th} time point (corresponding to LFP activity 50 ms in the past) was extracted. This procedure was repeated every 15 milliseconds, until four time points corresponding to the four previous inflection points in the LFP were extracted. Each subsequent inflection point measured in real time that exhibited a decreasing slope (i.e. points corresponding to peaks in the filtered LFP) set the arbitrary function generator to the on-state.

V. Gamma-Burst Optogenetic Stimulation

An arbitrary function generator (Agilent Technologies, 33210A) was set to the arbitrary waveform mode, with the stimulus signal calibrated to generate three 5 ms pulses with an inter-pulse-interval of 15 ms. Each LFP peak occurring in the real time sliding LFP window initiates a delay equivalent to the median time interval between last 5 LFP inflection points (that is, the median interval between the last five LFP peaks and troughs). After this delay, the function generator is placed in the ‘on’ state for 65 ms. The output from the function generator triggered a blue (473 nm wavelength, CrystaLaser, CL473-025-O) or yellow laser (593 nm wavelength, OEMLaser Systems, Inc., YL-593-00080-CWM-SD-05-LED-F). Light stimulation was delivered at 1.6 mW, and the laser output was measured using a Power meter (Thorlabs, PM100D). For the open loop control experiments, the average number of light pulses delivered to each animal using nCLASP during the first testing session was calculated. Control experiments were conducted in a group of TST-naïve mice stimulated with the same number of light pulses using an open-loop fixed frequency pattern (5 ms pulse width, 14.05 Hz stimulation).

W. Elastic Net Analysis

LFP power was calculated for each brain area and LFP coherence was calculated for each brain area pair. Data was averaged within each TST testing session, and resolved from 3 to 80 Hz in 1 Hz bins. This resulted in 2808 predictors per recording session, and a ‘difference matrix’ was calculated as the difference observed in each predictor between testing sessions. These 2808 difference measures were used as observation variables and the change in immobility across TST sessions were taken as the response at each observation. Since the sample size is much smaller than the number of the predictors, we resorted to penalized or regularized models. In particular, we used Elastic net, which induces sparsity while allowing grouped selection of predictors.

The Elastic Net is a regularized regression method that solves the problem:

$${min}_{{β}_{0} , β} ( \frac{1}{2 N} {\sum }_{i = 1}^{N} {( {y}_{i} - {β}_{0} - {x}_{i}^{T} β )}^{2} + λ {P}_{α} ( β ) )$$

$$where$$

$${P}_{α} ( β ) = \frac{( 1 - α )}{2} {‖ β ‖}_{2}^{2} + α {‖ β ‖}_{1} = {\sum }_{j = 1}^{p} ( \frac{( 1 - α )}{2} {β}_{j}^{2} + α \vert {β}_{j} \vert )$$

with N the number of observations, yᵢ the response at observation a vector of “feature” data (length p) at observation i, λ is a global positive regularization parameter, β₀ is a scalar “intercept”, β are coefficients on the different features, and alpha is a positive number which controls the tradeoff between the L1 and L2 penalty. The Elastic Net was solved using 10 fold cross validation, and optimized based on the global minimum ofthe mean squared error with alpha values ranging from 10̂-6 to 0.9 incremented on a logarithmic scale. Data was analyzed using the Matlab Machine Learning toolbox (The MathWorks, Inc., Natick, Mass.). Circular plots depicting neural predictors that were retained in the Elastic Net solution were generated using Circos.

X. Directionality of LFP Interactions

Directionality was inferred from LFP pairs based on the cross-correlation of instantaneous phases of oscillations at temporal lags. Similar approaches based on instantaneous amplitude correlations have been described in the literature. Briefly, LFP data acquired during the TST were filtered using Butterworth bandpass filters designed to isolate LFP oscillations within a 2 Hz window using a 1 Hz step (2-80 Hz). The instantaneous phase of the filtered LFPs were then determined using the Hilbert transform, and the instantaneous phase offset (ϕ_{Region1}−ϕ_{Region2})ₜ was calculated for the LFP time series. The mean resultant length (MRL) for the phase offset time series, corresponding to the deviation from circular uniformity (where 0 represents no deviation from circular uniformity and 1 represents a perfect distribution at a single angle/phase) was then calculated. Next, we introduced temporal shifts ranging from −250 ms to 250 ms in 2 ms increments into one of the LFP phase time series, and recalculated the MRL at each temporal offset. The temporal offset that yielded the optimal phase coupling was determined for each frequency band as the offset at which the highest MRL value was observed. Results were averaged across channels from the same brain region pairs for each animal. We also calculated directionality for the coherence measures retained in the Electome model using this approach. We calculated the 95% confidence interval for the optimal temporal offset of each LFP pair and frequency bin across animals. A frequency and brain area pair was deemed to exhibit significant directionality if the 95% confidence interval for the groups did not overlap with zero on either testing day (N=14 WT mice and 12 Clock-Δ19 mice). This approach has been previously utilized to quantify directionality across limbic neural circuits. All data in the text are presented as mean±s.e.m unless otherwise specified.

V. Resting Network Analysis

Video data was used to identify time segments during which animals were immobile (less than 4 cm/s instantaneous forward locomotion). Resting activity segments were then defined by at least 20 consecutive seconds of immobility that were preceded and followed by at least 5 seconds of immobility. Coherence was calculated from LFP pairs as described above.

VI. Conclusion

The particular arrangements shown in the Figures should not be viewed as limiting. It should be understood that other embodiments may include more or less of each element shown in a given Figure. Further, some of the illustrated elements may be combined or omitted. Yet further, an illustrative embodiment may include elements that are not illustrated in the Figures.

A step or block that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical functions or actions in the method or technique. The program code and/or related data can be stored on any type of computer readable medium such as a storage device including a disk, hard drive, or other storage medium.

The computer readable medium can also include non-transitory computer readable media such as computer-readable media that store data for short periods of time like register memory, processor cache, and random access memory (RAM). The computer readable media can also include non-transitory computer readable media that store program code and/or data for longer periods of time. Thus, the computer readable media may include secondary or persistent long term storage, like read only memory (ROM), optical or magnetic disks, compact-disc read only memory (CD-ROM), for example. The computer readable media can also be any other volatile or non-volatile storage systems. A computer readable medium can be considered a computer readable storage medium, for example, or a tangible storage device.

While various examples and embodiments have been disclosed, other examples and embodiments will be apparent to those skilled in the art. The various disclosed examples and embodiments are for purposes of illustration and are not intended to be limiting, with the true scope being indicated by the following claims.

1. A system comprising:
a stimulator;
a waveform generator communicatively coupled to the stimulator;
a data acquisition system; and
a controller comprising a memory and at least one processor, wherein the at least one processor is configured to execute instructions stored in the memory so as to carry out operations, the operations comprising:
receiving, via the data acquisition system, information indicative of an oscillatory signal from a first brain region;
determining, based on the received information, a reference phase of the oscillatory signal;
generating, by the waveform generator, at least one excitatory stimulus, wherein an onset of the generated at least one excitatory stimulus is based on the reference phase of the oscillatory signal; and
in response to the generated at least one excitatory stimulus, causing the stimulator to provide stimulation to a second brain region.

2. The system of claim 1, wherein the first brain region comprises a subgenual cingulate cortex and wherein the second brain region comprises a nucleus accumbens.

3. The system of claim 1, wherein the first brain region comprises a subgenual cingulate cortex and wherein the second brain region comprises an amygdala.

4. The system of claim 1, wherein the first brain region comprises a hippocampus and wherein the second brain region comprises a prefrontal cortex.

5. The system of claim 1, wherein the first brain region comprises a medial dorsal thalamus and wherein the second brain region comprises a ventral striatum.

6. The system of claim 1, wherein each excitatory stimulus of the at least one excitatory stimulus comprises a plurality of approximately 5 ms pulses having an inter-pulse interval of approximately 15 ms.

7. The system of claim 1, wherein the stimulator comprises a light source.

8. The system of claim 7, wherein the light source is configured to provide an excitatory optical stimulus that excites an optically-sensitive substance that is present in the second brain region.

9. The system of claim 1, wherein the stimulator comprises at least one electrode, and wherein the stimulator is configured to provide an excitatory electrical stimulus to the second brain region via the at least one electrode.

10. A method for delivering brain stimulation therapy to a patient in need thereof, the method utilizing the system of claim 1.

11. The system of claim 1, wherein each excitatory stimulus of the at least one excitatory stimulus has a duration that is less than half of a period of the oscillatory signal.

12. The system of claim 1, wherein each excitatory stimulus of the at least one excitatory stimulus has a duration that is less than one quarter of a period of the oscillatory signal.

13. The system of claim 1, wherein determining, based on the received information, a reference phase of the oscillatory signal comprises determining a reference phase of an oscillatory signal that is present in the received information and that has a frequency between 2 Hertz and 7 Hertz.

14. The system of claim 1, wherein the operations further comprise:
determining, based on the received information, that an oscillatory signal is present in the received information, wherein generating, by the waveform generator, at least one excitatory stimulus and causing the stimulator to provide stimulation to a second brain region are performed in response to determining that the oscillatory signal is present in the received information.

15. A method comprising:
detecting, from a first brain region, an oscillatory signal;
determining, based on the detected oscillatory signal, a reference phase of the oscillatory signal; and
providing, to a second brain region, at least one excitatory stimulus, wherein an onset of the at least one excitatory stimulus is based on the reference phase of the oscillatory signal.

16. The method of claim 15, wherein the first brain region comprises a subgenual cingulate cortex.

17. The method of claim 16, wherein the second brain region comprises a medial dorsal thalamus.

18. The method of claim 15, wherein the second brain region comprises a medial dorsal thalamus.

19. The method of claim 15, wherein detecting an oscillatory signal comprises detecting, using an electrode that is at least partially disposed within the first brain region, an electrical signal.

20. The method of claim 15, wherein providing an excitatory stimulus comprises providing the excitatory stimulus from a stimulator that is at least partially disposed within the second brain region.

21. The method of claim 15, wherein providing an excitatory stimulus comprises providing an excitatory optical stimulus.

22. The method of claim 15, wherein providing an excitatory stimulus comprises providing an excitatory electrical stimulus via one or more electrodes that are disposed at least partially within the second brain region.

23. The method of claim 15, wherein each provided excitatory stimulus comprises a respective plurality of excitatory pulses.
